(19) United States
(12) Patent Application Publication
(10) Pub. No.: US 2015/0151004 A1
(43) Pub. Date: Jun. 4, 2015

(54) METHODS AND COMPOSITIONS OF MODULATING TUMOR INITIATING CELLS AND THE USE THEREOF

(71) Applicants: Liang Xu, Lawrence, KS (US); Xiaojie Meng, Lawrence, KS (US); Xinbao Hao, Lawrence, KS (US)

(72) Inventors: Liang Xu, Lawrence, KS (US); Xiaojie Meng, Lawrence, KS (US); Xinbao Hao, Lawrence, KS (US)

(73) Assignee: THE UNIVERSITY OF KANSAS, Lawrence, KS (US)

(21) Appl. No.: 14/304,719

(22) Filed: Jun. 13, 2014

Related U.S. Application Data
(60) Provisional application No. 61/835,167, filed on Jun. 14, 2013.

(51) Int. Cl. A61K 47/48; A61K 31/713; C07K 16/28; A61K 9/127

(52) U.S. Cl. CPC A61K 47/48823; A61K 9/1272; A61K 31/713; A61K 47/48561; C07K 16/2884; A61K 48/00

(57) ABSTRACT

A therapeutic vector can include: a lipid particle; a CD44 antigen targeting moiety associated with the lipid particle; and a therapeutic nucleic acid associated with the lipid particle. The CD44 antigen targeting moiety can be anti-CD44 antibody and/or anti-CD44 scFv. The lipid particle can have a formula according to one of the following: DOTAP/DOPE 1:1 molar ratio; DDAB/DOPE 1:1 molar ratio; DDAB/DOPE 1:2 molar ratio; DOTAP/Chol 1:1 molar ratio; DDAB/Chol 1:1 molar ratio; DOTAP/DOPE/Chol 2:1:1 molar ratio; and DDAB/DOPE/Chol 2:1:1 molar ratio. The molar ratios can range +/−1%, 5%, 10%, or 20%. A method of inhibiting cancer development can include: providing the therapeutic vector and administering the therapeutic vector to the subject in an amount to inhibit cancer development.

### CROSS-REFERENCE TO RELATED APPLICATIONS

This patent application claims the benefit of U.S. Provisional Application No. 61/835,167 filed Jun. 14, 2013, which application is incorporated herein by specific reference in its entirety.

### GOVERNMENT SUPPORT STATEMENT

This invention was made with government support under R01 CA121830-0451 K-INBRE P20 GM103418 awarded by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Jun. 12, 2014, is named K1262.10037US02_SL.txt and is 2,498 bytes in size.

### BACKGROUND

It is thought that cancer stem cells are capable of self-renewal and differentiation as per identification in a variety of tumors. The cancer stem cells can be at least in colon, pancreas, prostate, brain, and breast cancers, but may be elsewhere. Accordingly, it may be advantageous to have systems and therapies for treating cancer stem cells.

### SUMMARY

In one embodiment, a therapeutic vector of the present invention can include: a lipid particle; a CD44 antigen targeting moiety associated with the lipid particle; and a therapeutic nucleic acid associated with the lipid particle. In one aspect, the CD44 antigen targeting moiety can be anti-CD44 antibody. In another aspect, the CD44 antigen targeting moiety can be anti-CD44 scFv. However, the vector can include both the anti-CD44 antibody and the anti-CD44 scFv, or a composition can have vectors with some having the antibody and/or some having the fragment.

In one embodiment, the lipid particle can be a liposome or other lipid particle. The lipid particle can have a formula according to one of the following: DOTAP/DOPE 1:1 molar ratio; DDAB/DOPE 1:1 molar ratio; DDAB/DOPE 1:2 molar ratio; DOTAP/Chol 1:1 molar ratio; DDAB/Chol 1:1 molar ratio; DOTAP/DOPE/Chol 2:1:1 molar ratio; and DDAB/DOPE/Chol 2:1:1 molar ratio. The molar ratios can range+/−1%, 5%, 10%, or 20%.

In one embodiment, the therapeutic nucleic acid can be selected from siRNA, siRNA mimics, miRNA, miRNA mimics, shRNA, shRNA mimics, plasmid DNA encoding therapeutic agent (e.g., genes, shRNA, miRNA, or the like), DNA fragment, shRNA expression cassette, miRNA expression cassette, RNA, oligonucleotides, or the like. In some examples, the therapeutic nucleic acid can be selected from Bcl-2 shRNA, pshBcl-2 plasmid, Mcl-1 shRNA, pshMcl-1 plasmid, miR-34, miR-34 mimic, or the like.

In one embodiment, a method of inhibiting cancer development in a subject can include: providing the therapeutic vector; and administering the therapeutic vector to the subject in an amount to inhibit cancer development. The inhibition can be prevention, which is likely in view of the efficiency and efficacy of the data; however, the inhibition can be a slowing or reduction in development compared to no treatment or compared to other known treatments, such as compared to other vectors that do not have the CD44 antigen targeting moiety. The data provides an indication that the therapeutic vector retards progression of cancer development. Accordingly, the therapeutic vector can target: precancerous cells; tumor initiating cells; cancer stem cells; progenitor cells, and/or any cells having the CD44 antigen. Pre-cancer stem cells have been shown to have the CD44 antigen.

In one embodiment, the method can include administering a sufficient amount of the therapeutic vector so as to reduce tumor initiation and/or tumor growth with the therapeutic nucleic acid. In one aspect, the therapeutic vector reduces tumor initiation and/or tumor growth with the therapeutic nucleic acid compared to no therapy or against a control or against a similar vector lacking the CD44 antigen targeting moiety.

In one embodiment, the therapeutic vector can be used to inhibit formation of tumorspheres, or significant reduction thereof. It is possible that once tumorspheres are forming, the nanovector can target the tumorspheres to provide the therapeutic nucleic acid to treat the tumorspheres and reduce the tumorspheres or kill the tumorspheres. The method can include administering a sufficient amount of the therapeutic vector so as to inhibit formation of tumorspheres with the therapeutic nucleic acid.

In one embodiment, the method can include administering a sufficient amount of the therapeutic vector so as to retard progression of cancer development with the therapeutic nucleic acid. The therapeutic vector can target precancerous cells, tumor initiating cells, progenitor cells, and/or cancer stem cells having the CD44 antigen so as to inhibit cancer development with the therapeutic nucleic acid.

In one embodiment, a method of preparing the therapeutic vector is provided. The method can include: providing the CD44 antigen targeting moiety with the lipid particle; associating the lipid particle with the CD44 antigen targeting moiety; and associating the therapeutic nucleic acid with the lipid particle. Also, the method can include providing the components of the lipid particle, and forming the lipid particle in the presence of the CD44 antigen targeting moiety. In one aspect, the method can include forming a lipid particle and CD44 antigen targeting moiety complex in the presence of serum free media. The method can include forming the therapeutic vector to have the following: (therapeutic nucleic acid DNA)/Lipid/(CD44 antigen targeting moiety) ratio in the range of 1/(0.1-50)/(0.1-100) μg/nmol/μg. Also, the method can include forming the therapeutic vector to have the following: (therapeutic nucleic acid miRNA or siRNA or the like)/Lipid/(CD44 antigen targeting moiety) ratio in the range of 1/(1-12)/(0.1-1.2) μg/nmol/μg.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes images showing β-gal (i.e., β-galactosidase) staining of the transfected cells with β-galactosidase expression vector at 0.2 μg/well, where blue cells have reporter LacZ gene expression.

FIG. 1B shows β-gal units per microgram protein for CD44mAb, CD44scFv, and the control (e.g., normal mouse IgG).

FIG. 1C shows β-gal units per microgram protein for CD44mAb, Tf, and the control (e.g., normal mouse IgG).

FIG. 2A includes images showing anti-CD44 antibody targeted nanovectors efficiently delivered GFP reporter gene to human pancreatic cancer MiaPaCa-2 tumorsphere-forming cells, where images were taken after 5 days of tumorsphere culture.

FIG. 2B includes a graph showing the GFP positive tumorspheres were counted under fluorescent microscopy for nano and anti-CD44-nano.

FIG. 3A includes images that show anti-CD44 antibody targeted nanovectors efficiently delivered GFP reporter gene to human pancreatic cancer BxPC-3 tumorsphere-forming cells, where images were taken after 5 days of tumorsphere culture.

FIG. 3B includes a graph showing the GFP positive tumorspheres were counted under fluorescent microscopy for nano and anti-CD44-nano.

FIG. 4A includes graphs showing total cells analysis of anti-CD44-nanovector delivered pGFP to CD44+/CD133+ cells in vitro, where MiaPaCa-2 cells were transfected with anti-CD44-nanovector-pGFP for 48 hours, trypsinized, and analyzed by flow cytometry, where anti-CD44 increased GFP+ cells.

FIG. 4B includes a graph showing MiaPaCa-2 cells were transfected with anti-CD44-nanovector-pGFP for 48 hours, trypsinized, and stained with APC-anti-CD44 and PE-anti-CD133, then analyzed by flow cytometry, where 2.29% cells are CD44+/CD133+.

FIG. 4C includes graphs showing GFP analysis in the CD44+/CD133+ cells, where anti-CD44 increased GFP+ cells from non-targeted nanovectors 13.5% to CD44-targeting nanovectors 30.9%.

FIG. 5A includes graphs showing anti-CD44-nanovector delivered FITC-siRNA to CD44+/CD133+ cells in MiaPaCa-2 xenograft tumors in vivo, where anti-CD44-nanovector containing FITC-siRNA (anti-CD44-Nano-siRNA) was injected i.v. into nude mice bearing MiaPaCa-2 xenografts, 8 mg/kg, twice at 0 and 12 hours, and the tumors were collected at 24 hours, treated with collagenase to get single cells, stained with APC-anti-CD44 and PE-anti-CD133, then analyzed by flow cytometry, where around 2% cells are CD44+/CD133+.

FIGS. 5B-5E include graphs showing GFP positive levels of tumors treated, where anti-CD44 antibody increased GFP+ cells in the tumor from non-targeted nanovectors 4.5% to CD44-targeting nanovectors 16.1%, a 4-fold improvement.

FIG. 6A includes data showing anti-CD44-nanovector delivered Bcl-2 shRNA inhibited cell growth and tumorsphere formation in human pancreatic cancer MiaPaCa-2 cells, where tumor cell growth curves of cells treated with nanovectors, were Bcl-2 shRNA delivered by anti-CD44-nanovector significantly inhibited tumor cell growth.

FIGS. 6B-6D include images and graphs that show tumorsphere formation of cells treated with nanovectors, where Bcl-2 shRNA delivered by anti-CD44-nanovector significantly inhibited tumorsphere formation (FIGS. 6B-6C) and tumorsphere growth (FIG. 6D).

FIG. 7A includes images that show anti-CD44-nanovector delivered Bcl-2 shRNA inhibited tumorsphere formation in human breast cancer MDA-MB-231 cells, where images of tumorspheres are from the cells treated with nanovectors, where Bcl-2 shRNA delivered by anti-CD44-nanovector significantly inhibited tumorsphere formation.

FIG. 7B includes a graph that shows anti-CD44-nanovector delivered Bcl-2 shRNA inhibited tumorsphere formation in human breast cancer MDA-MB-231 cells, where images of tumorspheres are from the cells treated with nanovectors, where Bcl-2 shRNA delivered by anti-CD44-nanovector significantly inhibited tumorsphere formation.

FIG. 7C includes a graph that shows anti-CD44-nanovector delivered Bcl-2 shRNA inhibited tumorsphere formation in human breast cancer MDA-MB-231 cells, where images of tumorspheres are from the cells treated with nanovectors, where Bcl-2 shRNA delivered by anti-CD44-nanovector significantly inhibited tumorsphere formation and tumorsphere growth.

FIG. 7D includes an image that shows enlargement of tumorspheres.

FIG. 8 includes data showing anti-CD44-nanovector delivered Bcl-2 shRNA inhibited tumor initiation of human pancreatic cancer MiaPaCa-2 in vivo in nude mice, illustrated by tumor growth curves.

FIG. 9A includes a graph showing anti-CD44-nanovector delivered Bcl-2 shRNA inhibited tumor initiation of human breast cancer MDA-MB-231 in vivo in nude mice, with tumor growth curves from the mice injected with 1×10⁶ cells.

FIG. 9B includes a graph showing anti-CD44-nanovector delivered Bcl-2 shRNA inhibited tumor initiation of human breast cancer MDA-MB-231 in vivo in nude mice, with tumor growth curves from the mice injected with 0.5×10⁶ cells.

FIG. 10A includes images showing anti-CD44-nanovector delivered miRNA miR-34a inhibited cell growth and tumorsphere formation in human pancreatic cancer MiaPaCa-2 cells, where the images are of tumorspheres from the cells treated with nanovectors, where miR-34a mimic delivered by anti-CD44-nanovector significantly inhibited tumorsphere formation.

FIG. 10B includes a graph showing anti-CD44-nanovector delivered miRNA miR-34a inhibited cell growth and tumorsphere formation in human pancreatic cancer MiaPaCa-2 cells, where the images are of tumorspheres from the cells treated with nanovectors, where miR-34a mimic delivered by anti-CD44-nanovector significantly inhibited tumorsphere formation.

FIG. 10C includes a graph showing anti-CD44-nanovector delivered miRNA miR-34a inhibited cell growth and tumorsphere formation in human pancreatic cancer MiaPaCa-2 cells, where the images are of tumorspheres from the cells treated with nanovectors, where miR-34a mimic delivered by anti-CD44-nanovector significantly inhibited tumorsphere formation and tumorsphere growth.

FIG. 11A includes images showing anti-CD44-nanovector delivered miRNA miR-34a mimic inhibited cell growth and tumorsphere formation in human breast cancer MDA-MB-231 cells, where tumorspheres from the cells were treated with nanovectors, where miR-34a mimic delivered by anti-CD44-nanovector significantly inhibited tumorsphere formation.

FIG. 11B includes a graph showing anti-CD44-nanovector delivered miRNA miR-34a mimic inhibited cell growth and tumorsphere formation in human breast cancer MDA-MB-231 cells, where tumorspheres from the cells were treated with nanovectors, where miR-34a mimic delivered by anti-CD44-nanovector significantly inhibited tumorsphere formation.

FIG. 11C includes a graph showing anti-CD44-nanovector delivered miRNA miR-34a mimic inhibited cell growth and tumorsphere formation in human breast cancer MDA-MB-231 cells, where tumorspheres from the cells were treated with nanovectors, where miR-34a mimic delivered by anti-CD44-nanovector significantly inhibited tumorsphere formation and tumorsphere growth.

FIG. 12 includes a graph showing anti-CD44-nanovector delivered miRNA miR-34a plasmid inhibited tumor initiation of human pancreatic cancer MiaPaCa-2 in vivo in nude mice.

FIGS. 13A-13B include graphs that show anti-CD44-nanovector delivered miRNA miR-34a mimic inhibited tumor initiation of human breast cancer MDA-MB-231 (FIG. 13A) and human prostate cancer PC-3 (FIG. 13B) in vivo in nude mice.

FIGS. 14A-14D include graphs that show radiation increased CD44+/CD133+ tumor initiating cells (TIC) or cancer stem cells (CSC).

FIGS. 15A-15B include graphs that show radiation increased transfection efficiency of anti-CD44-nanovectors in human pancreatic cancer cells in a radiation dose—(FIG. 15A) and time-dependent (FIG. 15B) manner, wherein FIG. 15A shows radiation dose-dependently increased transfection efficiency of anti-CD44-nanovectors, where human pancreatic cancer MiaPaCa-2 cells in 24-well plates were radiated at 0, 2, 4, and 8 Gy, after six hours, the cells were transfected by anti-CD44-nanovector-pCMVb, where 48 hours later, the cells were collected and lysed for quantitative β-galactosidase enzyme assay, where 4-8 Gy showed best increase of transfection efficiency of the anti-CD44-nanovectors, and where FIG. 15B shows radiation mediated increase of transfection efficiency of anti-CD44-nanovectors was dependent on the time between radiation and transfection, where the cells were radiated at 2 Gy, after 0, 6, 12, and 24 hours, the cells were transfected by anti-CD44-nanovector-pCMVb, where 48 hours later, the cells were collected and lysed for quantitative β-galactosidase enzyme assay, where treating cells six hours after radiation shows the best increase of transfection efficiency of the anti-CD44-nanovectors.

FIG. 16A includes a graph illustrating size for the different formulations of the mimic:lipA:CD44.

FIG. 16B includes a graph illustrating zeta potential for the different formulations of the mimic:lipA:CD44.

FIG. 16C shows an image of a gel illustrating optimization of anti-CD44-nanovectors formulation.

FIG. 17A includes images of cells showing optimization of anti-CD44-nanovectors formulation.

FIG. 17B includes an image of a gel showing optimization of anti-CD44-nanovectors formulation.

FIG. 18A includes graphs that show optimization of anti-CD44-nanovector to improve in vivo transfection efficiency to CD44+/CD133+ cancer stem cells, where anti-CD44-nanovectors containing FITC-siRNA (anti-CD44-nano-siRNA) or GFP plasmid (anti-CD44-nano-pGFP) were injected i.v. into nude mice bearing MiaPaCa-2 xenografts, and the tumors were collected, treated with collagenase to get single cells, stained with APC-anti-CD44 and PE-anti-CD133, then analyzed by flow cytometry, and where total cells analysis is of anti-CD44-nanovector delivery.

FIG. 18B includes a flow cytometry chart that illustrates 1.12% cells are CD44+/CD133+.

FIG. 18C includes graphs showing anti-CD44-nanovector delivery in the CD44+/CD133+ cells.

FIGS. 19A-19C include images of gels showing systemic Tf-nanovector-miR34a plasmid treatment downregulated target genes Bcl-2 and Notch1 expression in xenografts of human prostate cancer PC-3 (FIG. 19A), early passage human primary breast cancer UM2 (FIG. 19B), and early passage human primary pancreatic cancer (FIG. 19C).

FIGS. 19Da-19Dc include graphs that show qRT-PCR analysis of gene expression of PC-3 tumors in FIG. 19A.

FIG. 19E includes an image of a gel showing tf-nanovector-miR34a mimic systemic treatment inhibited target genes Bcl-2 and Notch1 expression in prostate cancer PC-3 xenografts, where mature miR34a levels in the treated PC-3 tumors were increased as measured by qRT-PCR.

FIG. 19F includes a graph showing tf-nanovector-miR34a mimic systemic treatment inhibited target genes Bcl-2 and Notch1 expression in prostate cancer PC-3 xenografts, where mature miR34a levels in the treated PC-3 tumors were increased as measured by qRT-PCR.

FIG. 20A includes an image of a gel showing anti-CD44-nanovector-delivered miRNA miR-34a downregulated target gene Bcl-2 expression in prostate cancer PC-3 xenografts, where systemic injection of anti-CD44-nanovectors carrying miR34a plasmid inhibited Bcl-2 expression correlates with the increased levels of miR34a as measured by qRT-PCR.

FIGS. 20Ba-20Bc include graphs showing anti-CD44-nanovector-delivered miRNA miR-34a downregulated target gene Bcl-2 expression in prostate cancer PC-3 xenografts, where systemic injection of anti-CD44-nanovectors carrying miR34a plasmid inhibited Bcl-2 expression correlates with the increased levels of miR34a as measured by qRT-PCR.

FIG. 20C includes an image of a gel showing anti-CD44-nanovector-delivered miRNA miR-34a downregulated target gene Bcl-2 expression in prostate cancer PC-3 xenografts, where systemic injection of anti-CD44-nanovectors carrying miR34a mimic inhibited Bcl-2 expression correlates with the increased levels of miR34a as measured by qRT-PCR.

FIGS. 20Da-20Db include graphs showing anti-CD44-nanovector-delivered miRNA miR-34a downregulated target gene Bcl-2 expression in prostate cancer PC-3 xenografts, where systemic injection of anti-CD44-nanovectors carrying miR34a mimic inhibited Bcl-2 expression correlates with the increased levels of miR34a as measured by qRT-PCR.

FIG. 21A includes a graph showing systemic miR34a delivery with Tf-nanovectors inhibits the growth of subcutaneous xenografts of human prostate cancer PC3, where athymic mice bearing subcutaneous PC3 xenografts were treated with either Tf-LipA-Vector(Nano-Vec) or Tf-LipA-miR-34a plasmid(Nano-pmiR34a) (n=10 mice per cohort) by tail-vein injection at a dose of 30 μg miRNA plasmid per mouse every other day (EOD), and were treatment with miR-34a nanovector significantly inhibited tumor growth (P<0.0001) compared with the negative control group.

FIG. 21B includes a graph showing systemic miR34a mimic (25 μg) delivered with Tf-nanovectors also inhibited tumor growth (P<0.0001).

FIG. 21C includes a graph showing miR-100, another tumor suppressor miRNA, delivered with Tf-nanovector, inhibited human breast cancer SUM 159 xenografts tumor growth by i.v. injection.

FIGS. 21D-21E include images that show the unique lamellar structure of Tf-nanovectors either with plasmid or mimic by TEM.

FIG. 22A includes a graph that shows systemic miR34a delivery with anti-CD44-nanovectors (e.g., aCD44-vector, aCD44-plasmid 34a) inhibits the growth of subcutaneous xenografts of human prostate and breast cancer, where athymic mice bearing subcutaneous PC3 xenografts were treated with either aCD44-LipA-Vector (Nano-Vec) or aCD44-LipA-miR-34a plasmid (Nano-pmiR34a) (n=10 mice per cohort) by tail-vein injection at a dose of 30 μg miRNA plasmid per mouse every other day (EOD), where treatment with miR-34a nanovector significantly inhibited tumor growth (P<0.0001) compared with the negative control group.

FIG. 22B includes a graph that shows systemic miR34a mimic (25 μg) delivered with aCD44-nanovectors also inhibited breast cancer SUM-159 xenografts tumor growth (P<0.0001).

FIG. 22C includes a graph that shows miR-100 delivered with aCD44-nanovector inhibited human breast cancer SUM 159 xenografts tumor growth by i.v. injection.

FIGS. 22D-22E include images that show the unique lamellar structure of aCD44-nanovectors either with plasmid or mimic by TEM and STEM. The STEM images at the lower panel show the unexpected ordered multi-lamellar nanostructure with a coating of aCD44 antibody on the outer surface, which provides a structural basis for the nanovectors' targeting function.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to systems and methods for treating, inhibiting, and/or preventing cancer development, initiation, differentiation, growth, migration, or the like. In one example, the systems and methods can be configured for inhibiting cancer stem cells from differentiating into cancer cells. The systems can include nanovectors and nanovectors compositions that can be used in treating, inhibiting, and/or preventing cancer. The nanovectors can be designed to specifically modulate tumor initiating cells, such as cancer stem cells and progenitor cells. The nanovectors can also be designed for preferential growth inhibition and/or death of the tumor initiating cells. The nanovectors can further be designed for sensitizing tumor initiating cells for preferential growth inhibition and/or death by the conventional therapies.

The nanovectors can be configured as a particle-based system. The particle nanovectors can be included in the compositions and the methods of use described herein. The particle nanovector systems can be configured to specifically modulate tumor initiating cells, including cancer stem cells and progenitor cells. The particle nanovectors can also be designed for preferential growth inhibition and/or death of the tumor initiating cells. The particle nanovectors can further be designed for sensitizing tumor initiating cells for preferential growth inhibition and/or death by the conventional therapies.

The nanovectors of the invention can be used with therapeutic protocols. The therapeutic protocols can be designed to use the nanovectors to specifically modulate tumor initiating cells, including cancer stem cells and progenitor cells. The therapeutic protocols can also be designed for inducing preferential growth inhibition and/or death of the tumor initiating cells. The therapeutic protocols can further be designed for inducing sensitization tumor initiating cells for preferential growth inhibition and/or death by the conventional therapies.

The nanovectors of the invention can be used in therapeutic systems that implement therapeutic protocols. The therapeutic systems can include diagnostic equipment, imaging equipment, nanovector delivery equipment, gene expression analysis equipment, gene silencing analysis equipment, or others. The therapeutic systems can be designed to deliver the nanovectors to specifically modulate tumor initiating cells, including cancer stem cells and progenitor cells. The therapeutic systems can also be designed to deliver the nanovectors for inducing preferential growth inhibition and/or death of the tumor initiating cells. The therapeutic systems can further be designed for delivering the nanovectors to induce sensitization tumor initiating cells for preferential growth inhibition and/or death by the conventional therapies.

As used herein, “nanovectors” refer generally to a nano-scale vector that releases a nucleic acid as a therapeutic agent, and is defined in accordance with the knowledge of one of ordinary skill in the art.

As used herein, “CD44” refers generally to the CD44 antigen, which is a cell-surface glycoprotein involved in cell-cell interaction and is a hyaluronate receptor that has been used extensively as a cellular marker for stem cells, and has been shown to be a useful marker for cancer stem cells of various types of cancer, and is defined in accordance with the knowledge of one of ordinary skill in the art. CD44 mediates adhesive cell-cell and cell-extracellular matrix interactions through binding to its main ligand, hyaluronan, a glycosaminoglycan highly concentrated in the endosteal region. Other ligands include osteopontin, fibronectin, and selectin, all of which are involved in cell trafficking and lodgement. Beyond its adhesion function, CD44 can also transduce multiple intracellular signal transduction pathways when ligated with hyaluronan or specific function-activating mAbs. CD44 is cell surface marker for cancer stem cells, and also can be used as targets for designing nanoparticle delivery systems targeting cancer stem cells, as shown herein.

As used herein, “microRNA” or “miRNA” refers generally to small non-coding RNA nucleic acids that function in transcriptional and post-transcriptional regulation of gene expression, and is defined in accordance with the knowledge of one of ordinary skill in the art. It is thought that miRNA are involved in RNA interference and gene silencing. The miRNAs regulate a variety of biological processes, including developmental timing, signal transduction, tissue differentiation and maintenance, disease, and carcinogenesis. Emerging evidence demonstrates that miRNAs also play an essential role in stem cell self-renewal and differentiation by negatively regulating the expression of certain key genes in stem cells. It has been shown that MicroRNA-21 knockdown disrupts glioma growth in vivo and displays synergistic cytotoxicity with neural precursor cell delivered S-TRAIL in human gliomas, and miRNA Let-7 regulates self-renewal of breast cancer stem cells. The miRNA miR-34 was identified as p53 target and a potential tumor suppressor. Over 50% of human cancers have mutant p53 and expression of miR-34a,b,c appears to be correlated with p53. miR-34 targets Notch, MET, and Bcl-2, genes involved in the self-renewal and survival of cancer stem cells. Studies show miR-34 potently inhibits mammosphere growth and tumor formation of p53−/− breast cancer cells, indicating the tumor suppressor miR-34 may hold significant potential as a therapeutic agent for breast cancer and other types of cancer, possibly by inhibiting cancer stem cells. Other miRNAs such as miR-15 and miR-16 have been reported to be able to down-regulate Bcl-2. They may be involved in tumor initiating cells or cancer stem cells survival. miR-100 is also involved in cancer stem cells self-renew. Accordingly, the nanovectors of the present invention can include any miRNA as a therapeutic agent, such as those described herein.

As used herein, “cancer stem cells” refer generally to precancerous stem cells that have the ability to undergo self-renewal and multi-lineage differentiation, and is defined in accordance with the knowledge of one of ordinary skill in the art. The cancer stem cells can include leukemic stem cells and cells bearing the phenotype ESA⁺CD44⁺CD24⁻ Lineage⁻ that have the properties of breast cancer stem cells. Cancer stem cells are able to be serially passaged in NOD/SCID mice, each time generating a stem cell population, as well as the more differentiated non-tumorigenic cells forming the bulk of the tumor. A feature of tumor stem cells is their relatively unlimited self-renewal capacity and their asymmetric cell division. These properties can contribute to tumor expansion, as well as account for tumor metastasis. Results demonstrate that Bmi-1 is also required for self-renewal of leukemic stem cells in the murine AML model. Cancer stem cells have up regulated Notch genes. The Notch signaling pathway is a short-range communication transducer that is involved in regulating many cellular processes (proliferation, stem cell and stem cell niche maintenance, cell fate specification, differentiation, and cell death) during development and renewal of adult tissues. Notch signaling has been highlighted as a pathway involved in the development of the breast and is frequently dysregulated in invasive breast cancer.

As used herein, “liposomes” refer generally to an artificial vesicle having a lipid bilayer that can be used for administration of agents to cells, and is defined in accordance with the knowledge of one of ordinary skill in the art.

As used herein, “nanoparticle” refers generally to a small object or ultrafine particle that behaves as a whole unit with respect to its transport and properties, such as sized from 1 to 100 nanometers, and is defined in accordance with the knowledge of one of ordinary skill in the art. Microparticles are larger than nanoparticles and are sized from 100 nanometers to 100 microns. Nanoparticles and microparticles can be prepared from ceramics, polymers, glass-ceramics, and material composites. The nanoparticles may also be configured as viral particles having a lipid capsid containing nucleic acids, where the lipid capsid can include proteomic targeting moieties.

As used herein, “drug delivery” refers generally to approaches, formulations, technologies, and systems for transporting a pharmaceutical compound, proteomic drug, nucleic acid, vector, or other active agent into a host or cell to achieve a desired therapeutic effect, and is defined in accordance with the knowledge of one of ordinary skill in the art.

In one embodiment, the present invention includes the use of polymeric, non-viral, or viral vector systems that are configured to deliver gene therapeutics to cancer patients. The vectors systems can be configured for high gene transfer efficiency and can include tumor targeting moieties. The non-viral vectors can include cationic liposomes configured to mediate gene transfer and may include a ligand recognized by a cell surface receptor. Receptor-mediated endocytosis represents a highly efficient internalization pathway in eukaryotic cells. The present invention can also include tumor-specific, ligand-targeting, self-assembled nanoparticle-DNA lipoplex systems that are designed for systemic gene therapy of cancer (see U.S. Pat. No. 6,749,863, which is incorporated herein by specific reference). In one aspect, a nanovector system can include a targeting moiety, such as transferrin (Tf) or a single chain antibody fragment (scFv) against the transferrin receptor, which is overexpressed in the majority of human cancers, including pancreatic cancer. The nanovector system can include TfR-scFv-targeted nanovectors. A nanoparticle lipoplex may generate nanovectors targeting cancer stem cells.

In one embodiment, the present invention includes compositions, systems, and methods of using a nanovector to deliver siRNA/miRNA to tumor initiating cells in order to knock down target genes responsible for self-renewal and/or survival of tumor initiating cells. The compositions, systems, and methods employing the nanovector can lead to impaired tumorigenesis, tumor growth inhibition, reduced metastasis, and/or increased sensitivity to conventional chemo/radiotherapy.

In one embodiment, the nanovectors described herein can be prepared by a method that involves self-assembly of the nanovectors. The components of the self-assembled nanovectors can be used as a therapy for targeting and inhibiting human tumor initiating cells, where the components are delivered to tumor initiating cells and the nanovectors self-assemble to provide the therapy. Studies show the siRNA and/or miRNA and/or other therapeutics can be delivered by the self-assembled nanovectors to provide a therapy against human tumor initiating cells. The therapy can include tumor-targeted silencing of the genes critical for cancer progression, survival, resistance, and metastasis. The therapy can target tumor initiating cells, which are responsible for the recurrence, metastasis, and resistance to current cancer treatment and therapies.

In one embodiment, the nanovectors described herein can be prepared into therapeutic products that are used to treat patients with cancer, or as an adjuvant therapy to improve chemo and/or radiation cancer therapy. The nanovector therapeutic products can be configured to deliver siRNA/miRNA to tumor initiating cells and successfully knock down target genes responsible for the self-renewal and/or survival of such tumor initiating cells. The nanovector therapeutic products can provide therapies, such as impaired tumorigenesis, tumor growth inhibition, reduced metastasis, and/or increased sensitivity to conventional chemo/radiotherapy. The improvement in therapy can be compared to patients or cells where there is no therapy or conventional chemo/radiotherapy or in patients desensitized thereto.

In one embodiment, the nanovector can target the CD44 antigen and provide a siRNA for RNA interference of genes involved in tumor differentiation, tumorigenesis, tumor growth, metastasis, or other cancer development. The nanovector can be a CD44-targeting siRNA-nanovector. The nanovectors can be configured to include: (1) anti-CD44 antibody as a targeting moiety that specifically directs nanovectors to the CD44+ tumor initiating cells or cancer stem cells and thus efficiently deliver drugs or siRNA/miRNA or other active agents to the target cells; and (2) siRNA/miRNA that is delivered by the anti-CD44-nanovectors can silence the target genes responsible for cancer stem cells self-renewal, and block the dysregulated self-renewal of such cells. Although CD44 is not a specific marker for cancer stem cells, the specificity of the inventive targeted siRNA-nanovectors is based at least upon: (1) tumor initiating cells or cancer stem cells are CD44+ and depend on genes (e.g., hedgehog pathway: Gli-1/Bmi-1; Notch pathway) for excessive/dysregulated self-renewal or for survival/growth (Bcl-2, cMET) (e.g., oncogene addiction); and (2) normal CD44+ stem cells have no such gene over-activation and thereby will not be affected by siRNA/miRNA (e.g., therapeutic window). The combination of specific targeting to certain cells and targeting certain biological pathways can provide the specificity of the therapy being preferentially toward tumor initiating cells or cancer stem cells.

In one embodiment, the methods of the present invention can also include protocols for identifying the presence of the tumor initiating cells, such as cancer stem cells and progenitor cells, in a subject. The identification can include using CD44 as a target for identifying the presence of these types of cells that are pre-cancerous. After identification of these types of cells, the vectors and compositions of the present invention can be used in a therapy to inhibit the transformation thereof into cancer cells or a tumor. Such inhibition can be a reduction in the transformation rate, slowing of differentiation rate, slowing of growth rate, slowing of tumor development, or the like that inhibit or slows cancer progression. Such inhibition or slowing can be in comparison to situations when the inventive vectors and compositions are not utilized.

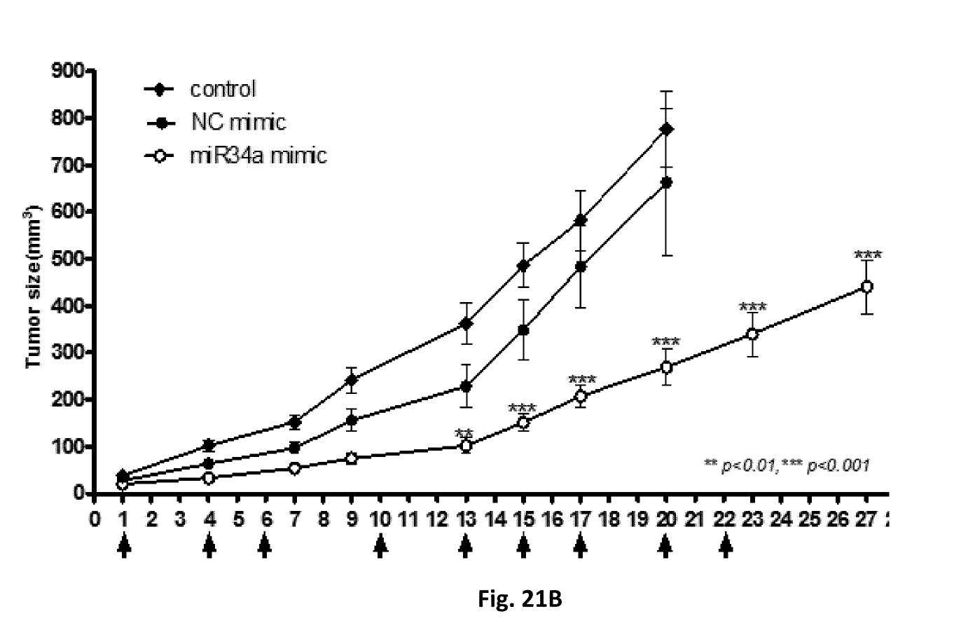

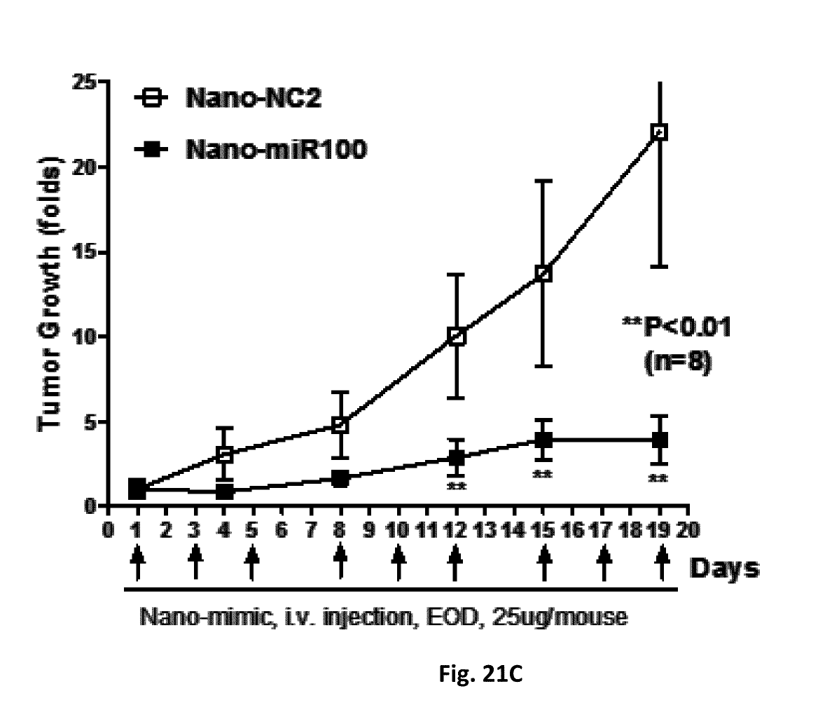

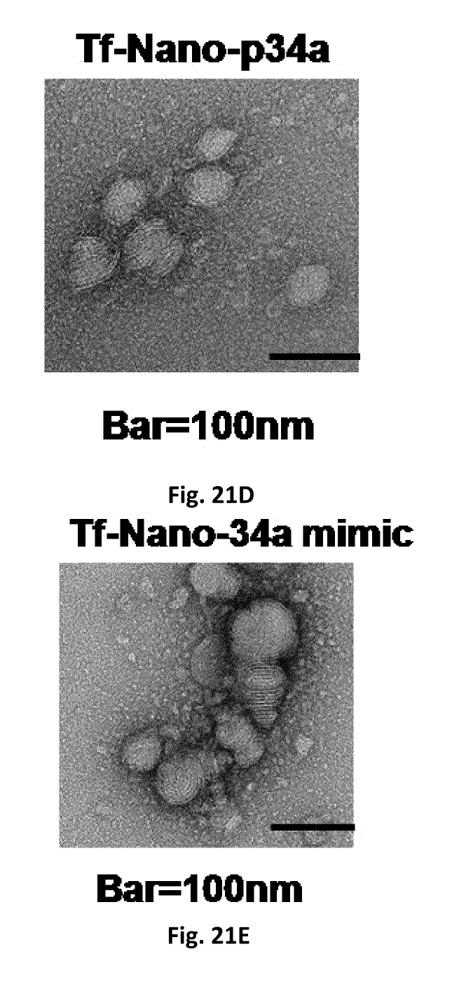

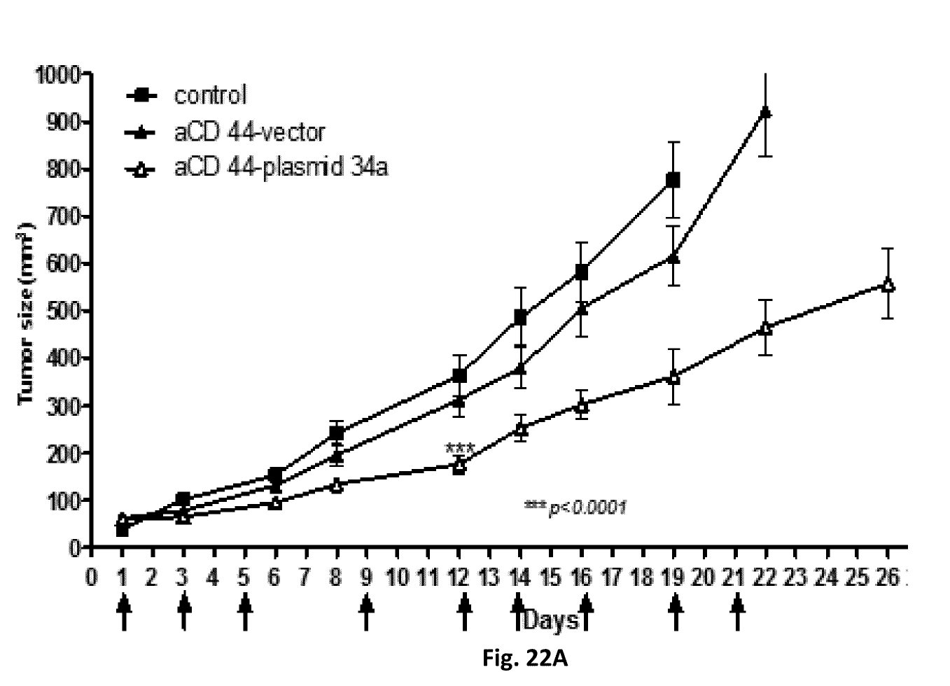

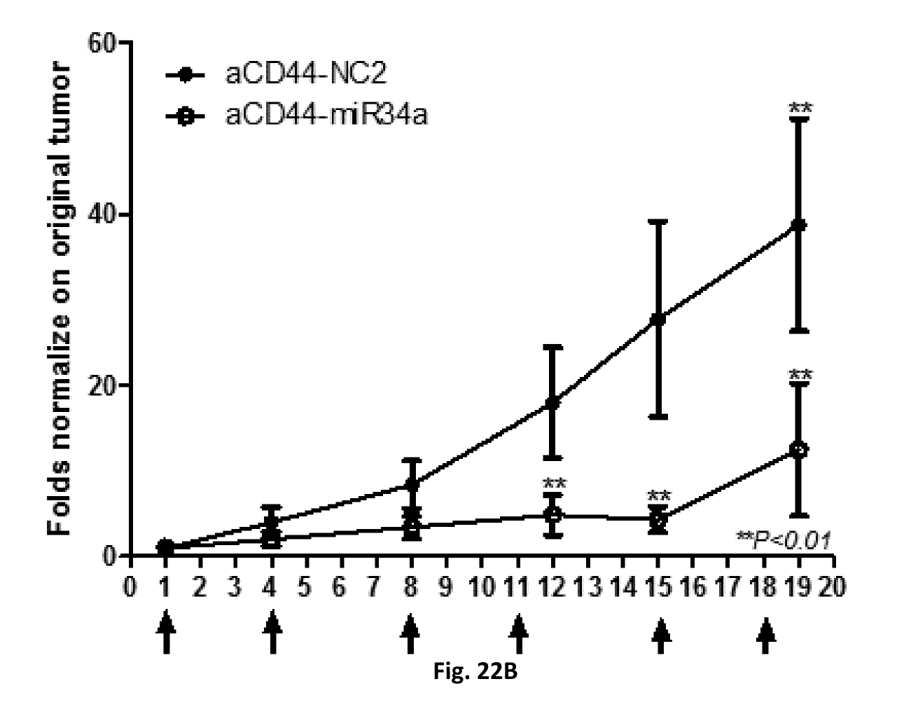

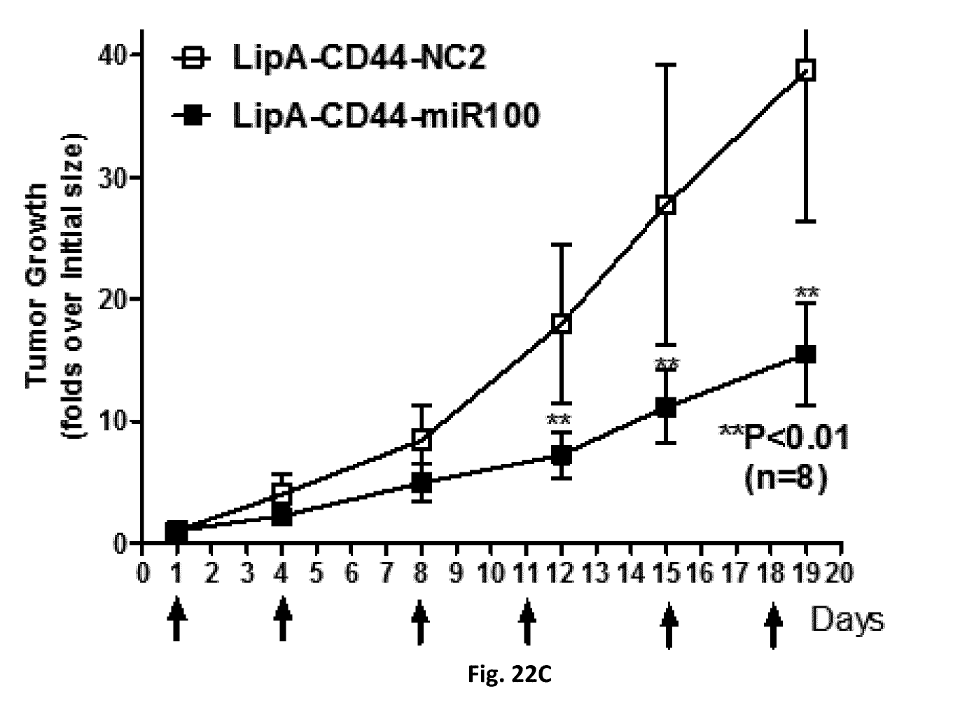

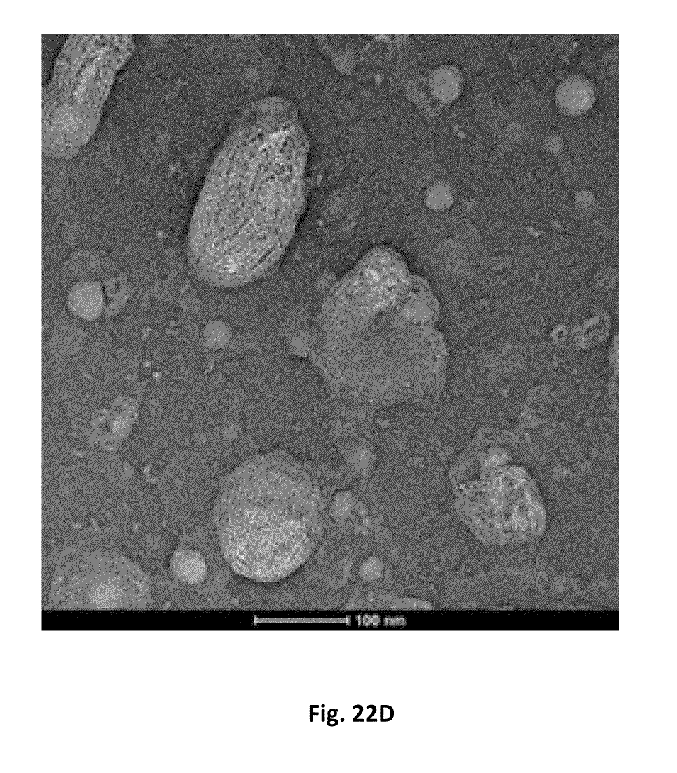

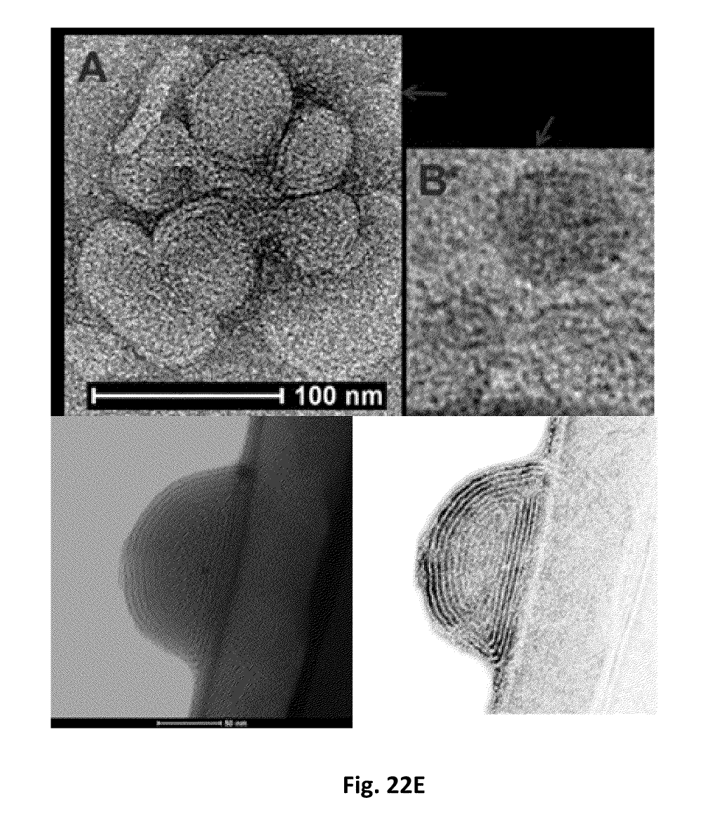

It has been determined that the present invention using the inventive vectors and compositions can provide surprising and unexpected improvement of in vivo efficacy (e.g., overall inhibition of cancer development), efficiency (e.g., percentage of cancer stem cells receiving the nanovector therein), and specificity compared to current or prior technologies or platforms. Targeting the tumor initiating cells has heretofore been exceedingly difficult with significantly lower in vivo efficacy, efficiency, and specificity; however, now with the present inventive vectors and compositions the in vivo efficacy, efficiency, and specificity are now substantially increased. Surprising and unexpectedly, it has been found that the in vivo efficiency of the inventive vectors is at least about 60% (e.g., 66%) of the tumor initiating cells, such as cancer stem cells. This is significant over the prior technologies. The surprising and unexpected results are, in part, described in Examples 10, 11, and 20A-20B as well as FIGS. 21-22. The cryo-EM and STEM images show the unexpected ordered multi-lamellar nanostructure with a coating of aCD44 antibody on the outer surface, which provides a structural basis for the nanovectors' targeting function to the CD44+ cancer stem cells.

In one embodiment, the nanovector described herein having the CD44 targeting moiety can deliver any type of nucleic acid, which nucleic acids can be single stranded, double stranded, or other known arrangement. The nucleic acid size can vary from small oligonucleotides to siRNA to miRNA, shRNA, plasmids, or precursors thereof or nucleic acids encoding for the production of the same. The nanovector can be used in gene therapy. The nucleic acids can be any type of nucleic acid that is therapeutic in order to inhibit or otherwise slow cancer development or progression.

In one embodiment, the nanovector of the present invention includes the liposome or other possible lipid particle having the formulations provided herein, which nanovector includes the CD44 targeting moiety for targeting cancer stem cells. The CD44 targeting moiety can be the CD44 antibody or fragment or portion thereof, such as a CD44 scFv fragment. CD44 antibody and CD44 scFV are known in the art. Surprisingly and unexpectedly, the CD44 targeting moiety nanovector described herein is significantly better than a vector having the transferrin targeting moiety or similar targeting moiety.

In one embodiment, a therapeutic vector of the present invention can include: a lipid particle; a CD44 antigen targeting moiety associated with the lipid particle; and a therapeutic nucleic acid associated with the lipid particle. In one aspect, the CD44 antigen targeting moiety can be anti-CD44 antibody. In another aspect, the CD44 antigen targeting moiety can be anti-CD44 scFv. However, the vector can include both the anti-CD44 antibody and the anti-CD44 scFv, or a composition can have vectors with some having the antibody and/or some having the fragment. The anti-CD44 antibody and fragment thereof can be simply referred to as a CD44 antibody or CD44 fragment to understand that the CD44 antibody or fragment targets the CD44 antigen. Any number of such CD44 antigen targeting moieties can be included on each lipid particle. It is possible that a single targeting moiety can be used on each particle; however, more than one targeting moiety can be used per lipid particle.

In one embodiment, the lipid particle can be a liposome or other lipid particle. The lipid particle can have a formula according to one of the following: DOTAP/DOPE 1:1 molar ratio; DDAB/DOPE 1:1 molar ratio; DDAB/DOPE 1:2 molar ratio; DOTAP/Chol 1:1 molar ratio; DDAB/Chol 1:1 molar ratio; DOTAP/DOPE/Chol 2:1:1 molar ratio; and DDAB/DOPE/Chol 2:1:1 molar ratio. However, other ratios and lipid members can be used. For example, the DOTAP/DOPE can range from 10:1 to 1:10 molar ratio; the DDAB/DOPE can range from 10:1 to 1:10 molar ratio; the DDAB/DOPE can range from 10:2 to 2:10 molar ratio; the DOTAP/Chol can range from 10:1 to 1:10 molar ratio; the DDAB/Chol can range from 10:1 to 1:10 molar ratio; the DOTAP/DOPE/Chol can range from 20:1:1 to 2:10:1 to 2:1:10 molar ratio; and DDAB/DOPE/Chol can range from 20:1:1 to 2:10:1 to 2:1:10 molar ratio. However, other ratios and lipid members can be used.

In one embodiment, the DOTAP/DOPE can range from 1:(0.5 to 3), or 1:(0.8 to 2.5), preferably 1:(1 to 2) molar ratio; the DDAB/DOPE can range from 1:(0.5 to 3), or 1:(0.8 to 2.5), preferably 1:(1 to 2) molar ratio; the ratio of cationic lipid to neutral lipid is about 1:(0.5-3), preferably 1:(1-2) (molar ratio). The ligand can be bound or mixed with cationic lipid and neutral lipid at a molar ratio of about (0.1 to 20):100, preferably (1 to 10):100, and more preferably (2.5 to 5):100 (ligand-lipid:total lipids), respectively. However, other ratios and lipid members can be used.

In one embodiment, the therapeutic nucleic acid can be selected from siRNA, siRNA mimics, miRNA, miRNA mimics, shRNA, shRNA mimics, plasmid DNA encoding therapeutic agent (e.g., genes, shRNA, miRNA, or the like), DNA fragment, shRNA expression cassette, miRNA expression cassette, RNA, oligonucleotides, or the like. In some examples, the therapeutic nucleic acid can be selected from Bcl-2 shRNA, pshBcl-2 plasmid, Mcl-1 shRNA, pshMcl-1 plasmid, miR-34, miR-34 mimic, or the like. In one aspect, the nanovector can include two or more different therapeutic nucleic acids, which can be of the same type (e.g., siRNA) or different types (e.g., siRNA and miRNA or siRNA and plasmid, or other combination). The nanovector can include one or more therapeutic nucleic acids per lipid particle, where multiple therapeutic nucleic acids can be included in each lipid particle.

In one embodiment, a method of inhibiting cancer development in a subject can include: providing the therapeutic vector and administering the therapeutic vector to the subject in an amount to inhibit cancer development. The inhibition can be prevention, which is likely in view of the efficiency and efficacy of the data; however, the inhibition can be a slowing or reduction in development compared to no treatment or compared to other known treatments, such as compared to other vectors that do not have the CD44 antigen targeting moiety. The data provides an indication that the therapeutic vector retards progression of cancer development. Accordingly, the therapeutic vector can target: precancerous cells, tumor initiating cells, cancer stem cells, progenitor cells, and/or any cells having the CD44 antigen. Pre-cancer stem cells have been shown to have the CD44 antigen. It should be recognized that the nanovector can be formulated into any suitable formulation for any suitable route of administration to a subject. The administration can be in vivo administration into the body of a subject, such as intravenous administration.

In one embodiment, the method can include administering a sufficient amount of the therapeutic vector so as to reduce tumor initiation and/or tumor growth with the therapeutic nucleic acid. In one aspect, the therapeutic vector reduces tumor initiation and/or tumor growth with the therapeutic nucleic acid compared to no therapy or against a control or against a similar vector lacking the CD44 antigen targeting moiety.

In one embodiment, the therapeutic vector can be used to inhibit formation of tumorspheres, or significant reduction thereof. It is possible that once tumorspheres are forming, the nanovector can target the tumorspheres to provide the therapeutic nucleic acid to treat the tumorspheres and reduce the tumorspheres or kill the tumorspheres. The method can include administering a sufficient amount of the therapeutic vector so as to inhibit formation of tumorspheres with the therapeutic nucleic acid.

In one embodiment, the method can include administering a sufficient amount of the therapeutic vector so as to retard progression of cancer development with the therapeutic nucleic acid. The therapeutic vector can target precancerous cells, tumor initiating cells, progenitor cells, and/or cancer stem cells having the CD44 antigen so as to inhibit cancer development with the therapeutic nucleic acid.

In one embodiment, a method of preparing the therapeutic vector is provided. The method can include: providing the CD44 antigen targeting moiety with the lipid particle; associating the lipid particle with the CD44 antigen targeting moiety; and associating the therapeutic nucleic acid with the lipid particle. Also, the method can include providing the components of the lipid particle and forming the lipid particle in the presence of the CD44 antigen targeting moiety. In one aspect, the method can include forming a lipid particle and CD44 antigen targeting moiety complex in the presence of serum-free media.

The method can include forming the therapeutic vector to have the following: (therapeutic nucleic acid DNA)/Lipid/(CD44 antigen targeting moiety) ratio in the range of (0.1 to 10)/(0.1 to 50)/(0.1 to 100), preferably (1 to 10)/(1 to 24)/(0.1 to 10), and more preferably 1/(1 to 12)/(0.1 to 1.2) μg/nmol/μg, respectively.

### Examples

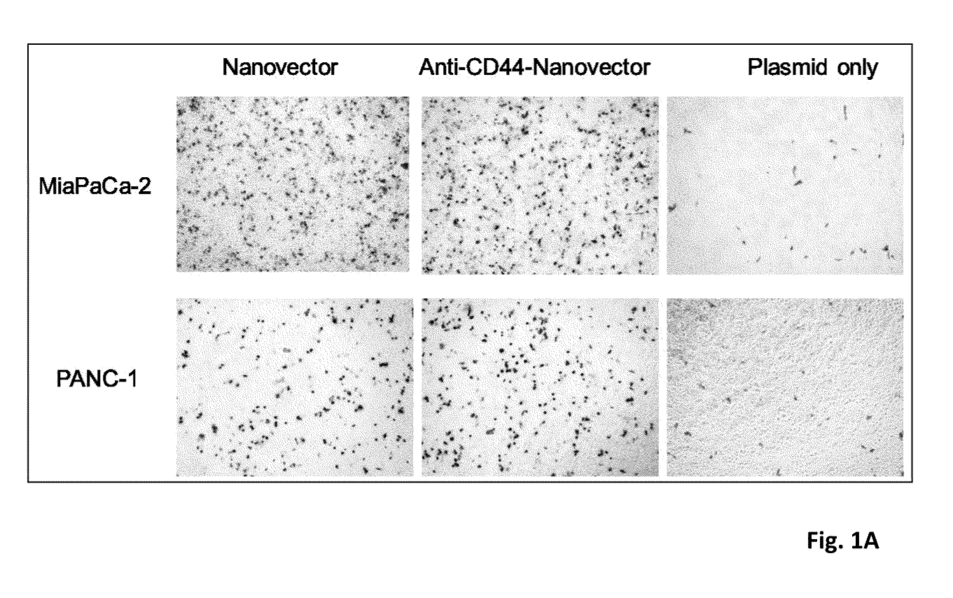

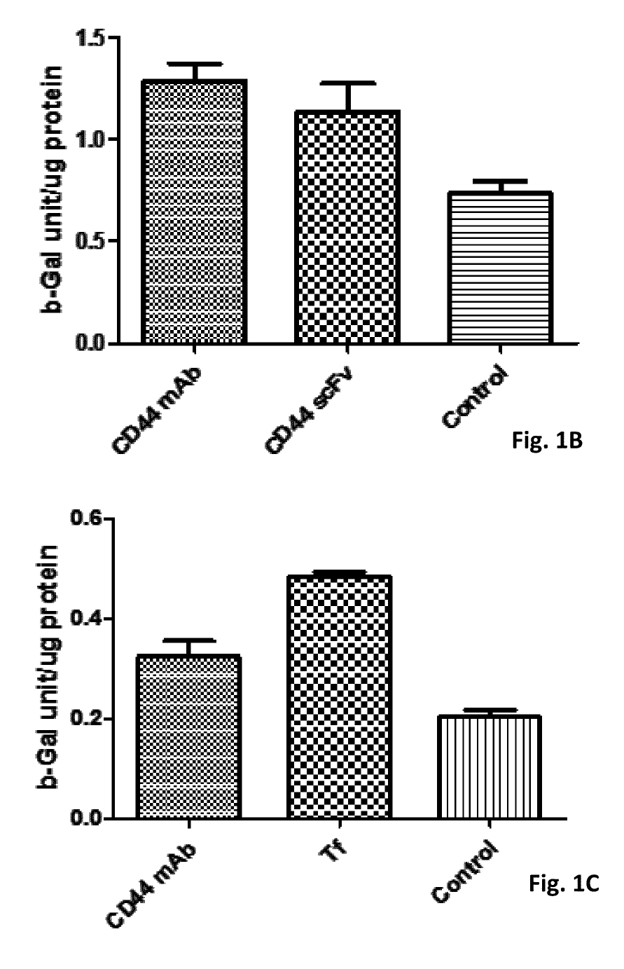

It has been found that anti-CD44 monoclonal antibody or scFv improves transfection efficiency of nanovectors in cancer cells. We have employed anti-CD44 monoclonal antibody (mAb) and anti-CD44 scFv in liposome-DNA complex to make nanovectors, using the method modified from that with transferrin or scFv to transferrin receptor as generally known in the art. We used beta-galactosidase as a reporter gene, driven by CMV, as generally known. Briefly, anti-CD44 CD44 mAb or anti-CD44 scFv were first mixed with DOTAP:DOPE (1:1) cationic liposome (2 mM) at a protein/lipid w/w ratio around 1:30. Then the complex was mixed with β-galactosidase expression plasmid (pCMVb) at a DNA/lipid ratio of 1 μg/10 nmol. Human pancreatic cancer MiaPaCa-2 and Panc-1 cells were transfected as described and 48 hours later, cells were either stained for beta-Gal or lysed for β-galactosidase enzyme activity. As shown in FIGS. 1A-1C, anti-CD44 antibody and scFv improves transfection efficiency of nanovectors in human pancreatic cancer cells. Other cancer cells were also tested and showed similar results.

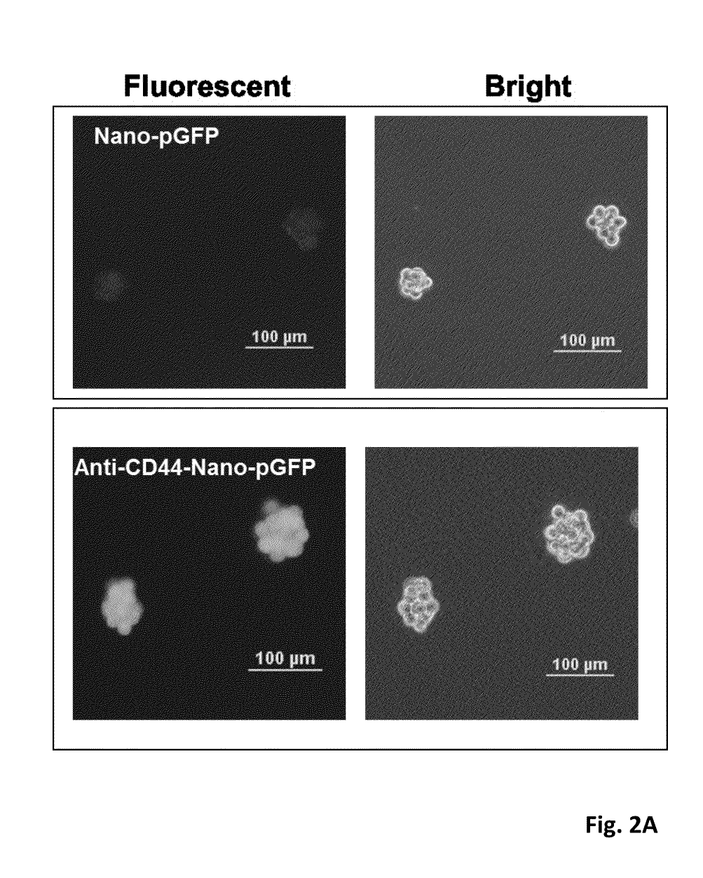

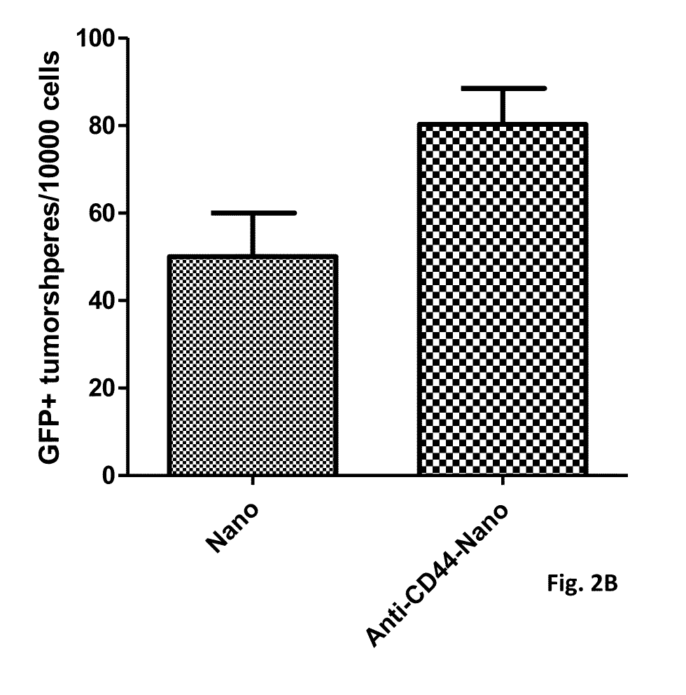

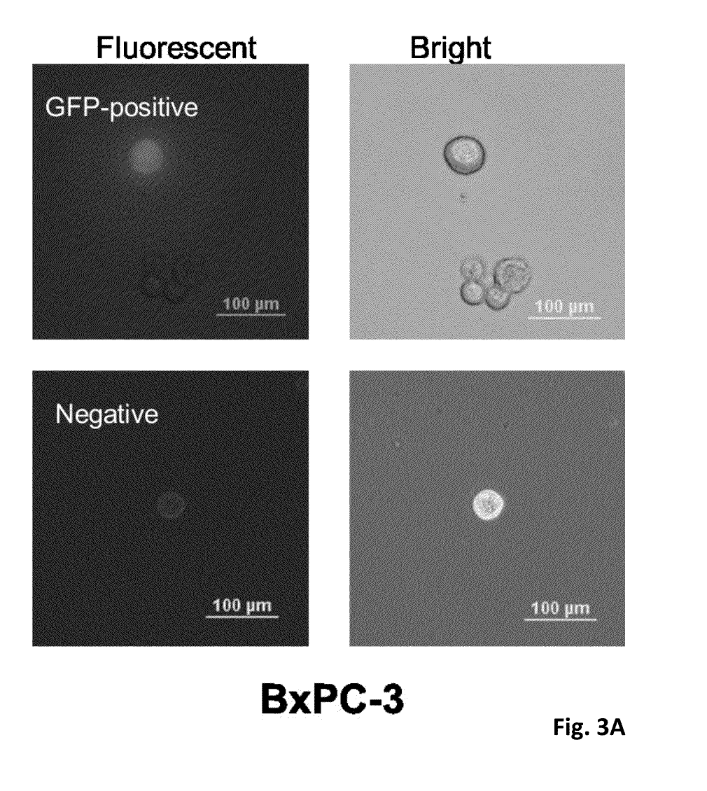

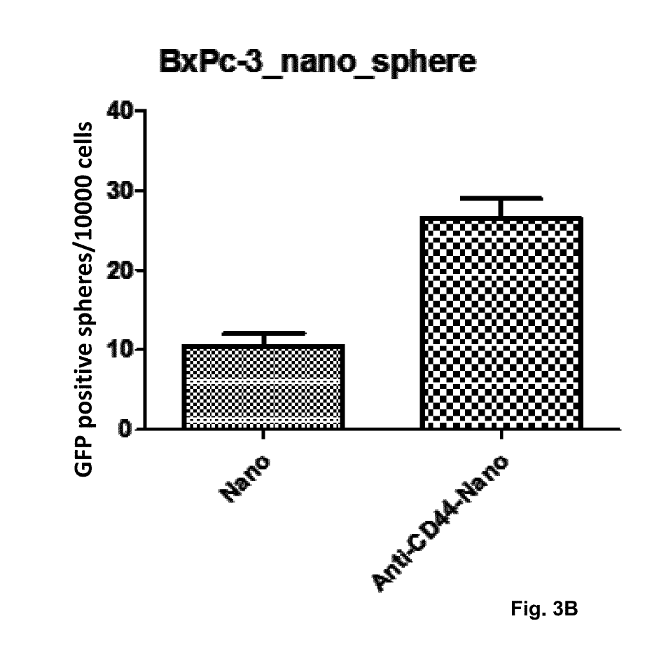

It has been found that anti-CD44 antibody or scFv targeted nanovectors efficiently delivered reporter genes to tumorsphere-forming cells. Tumorsphere culture is a widely used assay to assess the stem cells self-renewal and anchorage-independent growth. We employed green fluorescence protein (GFP) gene as a reporter gene, transfected cancer cells, and examined whether anti-CD44 antibody targeted nanovectors can deliver genes to tumorsphere-forming cells. Briefly, cells were transfected with anti-CD44-nanovectors carrying GFP expression plasmid (pGFP), as described above. 24-48 hours later, the cells were digested with 0.05% trypsin for five minutes and seeded in ultra-low affinity six-well plates with density of 8,000 cells per well, in 2 ml serum-free medium supplemented with EGF and FGF growth factors. As shown in FIGS. 2A-2B, anti-CD44-nanovectors efficiently delivered GFP reporter gene to human pancreatic cancer MiaPaCa-2 tumorsphere-forming cells, the whole tumorsphere showed strong green fluorescence. Similar results were also observed in BcPC-3 cells (see FIGS. 3A-3B). Other cancer cells were also tested and showed similar results.

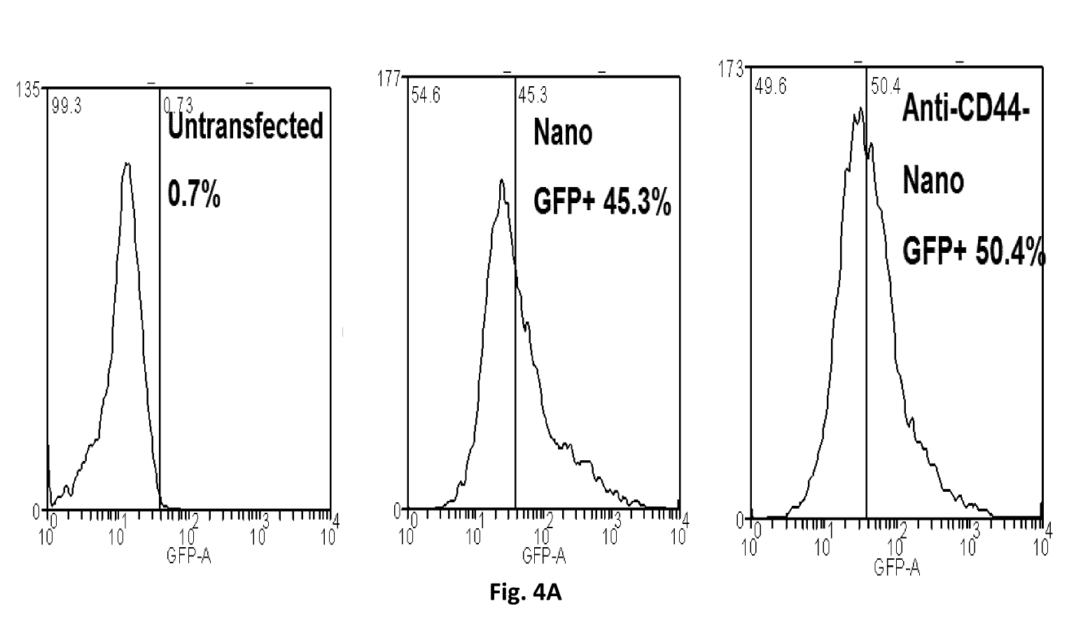

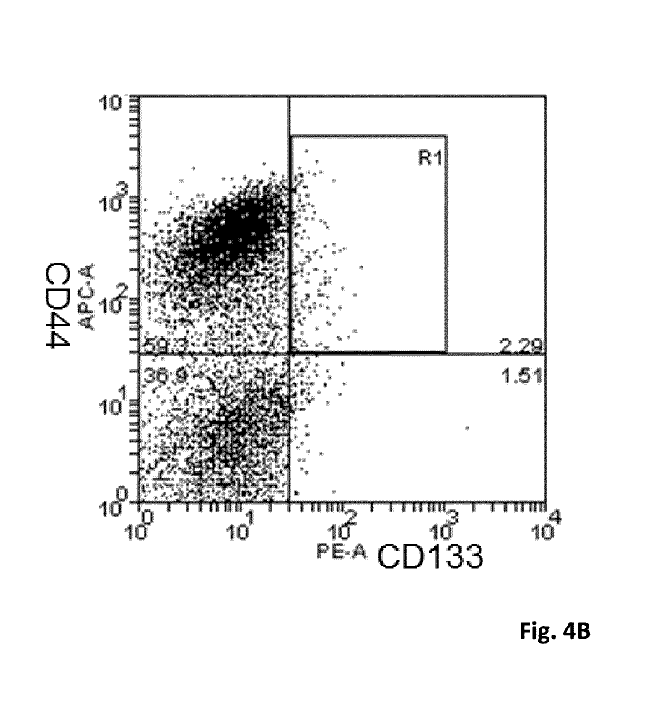

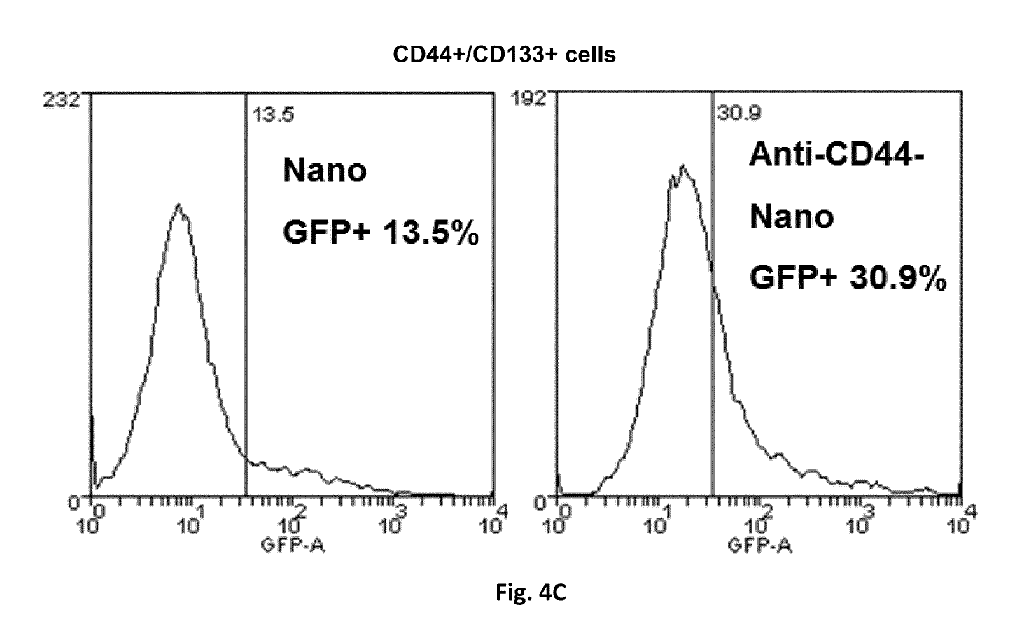

It has been found that anti-CD44 antibody or scFv targeted nanovectors efficiently delivered reporter genes/siRNA/miRNA to CD44+/CD133+ cancer cells both in vitro and in vivo. We have used flow cytometry to analyze the anti-CD44-nanovector targeted delivery. MiaPaCa-2 cells were transfected with anti-CD44-nanovector-pGFP for 48 hours, trypsinized, and analyzed by flow cytometry. As shown in FIG. 4A, anti-CD44 increased GFP+ cells from non-targeted nanovectors 45.3% to CD44-targeting nanovectors 50.4%. To assess whether anti-CD44-nanovectors can deliver contents to cancer stem cell population, the transfected cells were also stained with APC-anti-CD44 and PE-anti-CD133, then analyzed by flow cytometry. As shown in FIG. 4B, 2.29% cells are CD44+/CD133+, a population considered to be cancer stem cells. In sorted cells tumorsphere formation assay, the CD44+/CD133+ double-positive cells produced 30 tumorspheres per 3,000 cells, versus CD44+ or CD133+ single positive cells (3-5 tumorspheres per 3,000 cells), while CD44−/CD133− double negative cells had no tumorspheres. As shown in FIG. 4C, in this double-positive CD44+/CD133+ tumor stem cells population, anti-CD44 increased GFP+ cells from non-targeted nanovectors 13.5% to CD44-targeting nanovectors 30.9%, almost 2.3-fold increase due to anti-CD44 antibody targeting. The data provide support that anti-CD44-nanovectors can indeed deliver their contents to cancer stem cells.

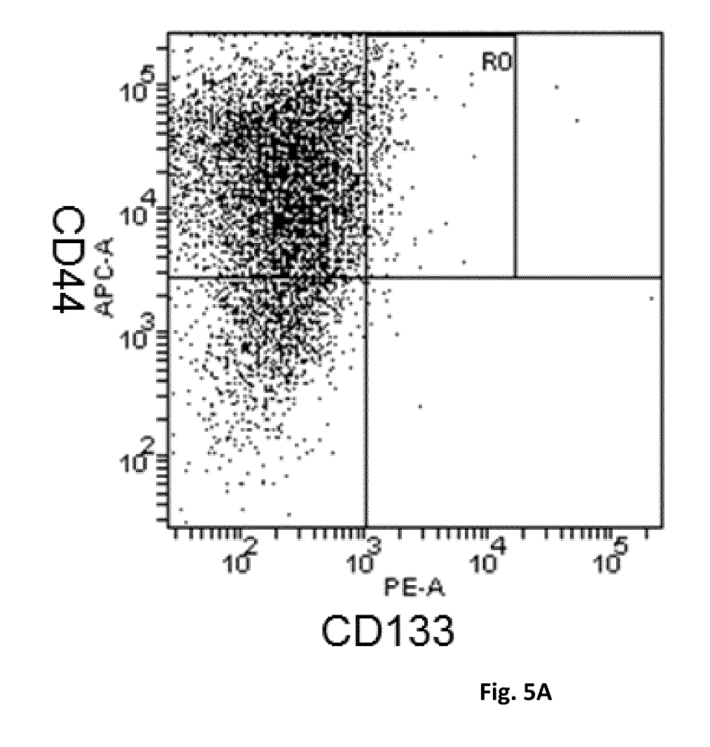

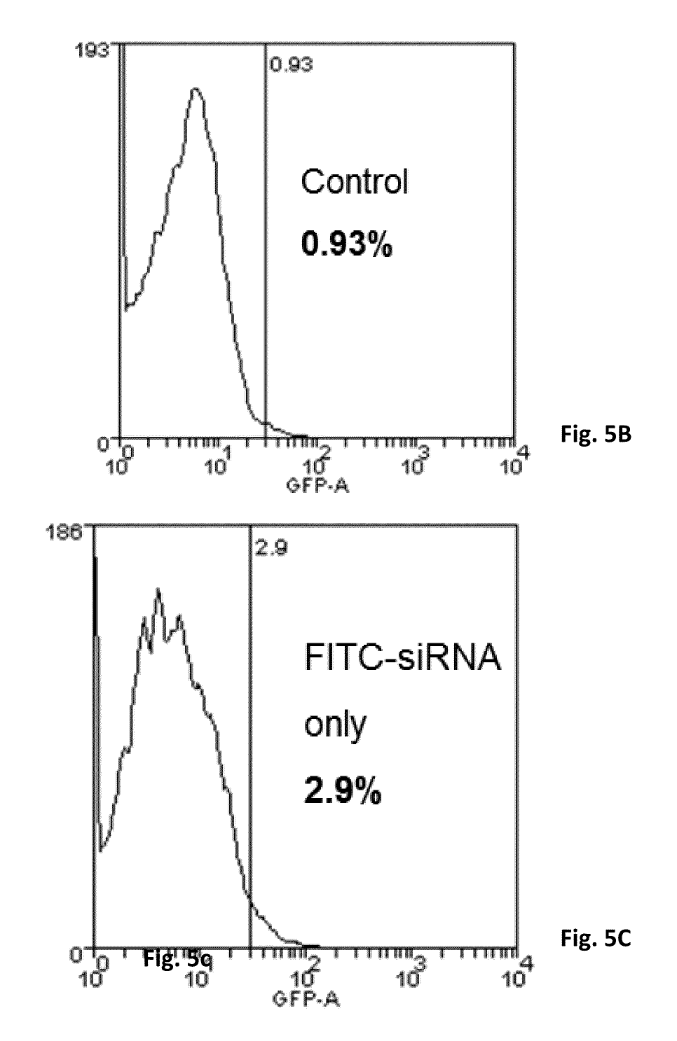

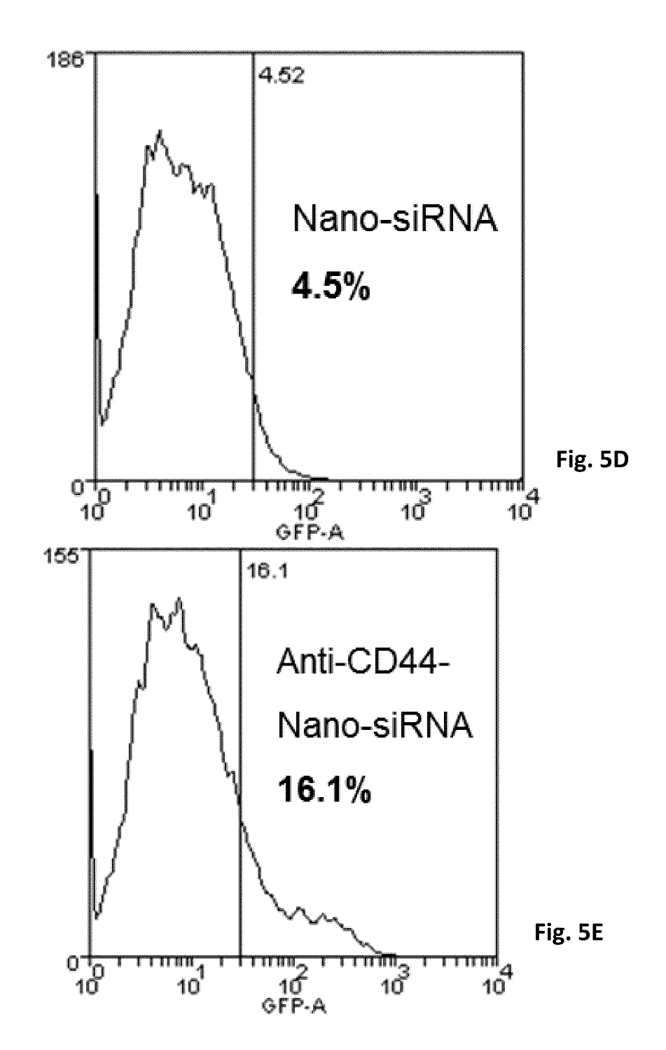

To evaluate the in vivo cancer stem cells targeted delivery by anti-CD44-nanovectors, we carried out in vivo assay in MiaPaCa-2 xenograft tumor model in nude mice. Anti-CD44-nanovector containing FITC-siRNA (e.g., random sequences) (anti-CD44-Nano-siRNA) was injected i.v. into nude mice bearing MiaPaCa-2 xenografts, 8 mg/kg, twice (0 and 12 hours), and the tumors were collected at 24 hours, treated with collagenase to obtain single cells, stained with APC-anti-CD44 and PE-anti-CD133, then analyzed by flow cytometry. As shown in FIG. 5A, around 2% cells are CD44+/CD133+, a population considered to be cancer stem cells. In this CD44+/CD133+ double-positive tumor stem cells population, anti-CD44 antibody increased GFP+ cells in the tumor from non-targeted nanovectors 4.5% to CD44-targeting nanovectors 16.1% (FIG. 5B-E), almost fourfold improvement due to the anti-CD44 antibody targeting. The data provide strong support that anti-CD44-nanovectors can indeed deliver siRNA to cancer stem cells in vivo. The absolute efficiency for cancer stem cells delivery by anti-CD44-nanovectors can be further improved by optimizing the compositions of the nanovectors and by multiple i.v. injections.

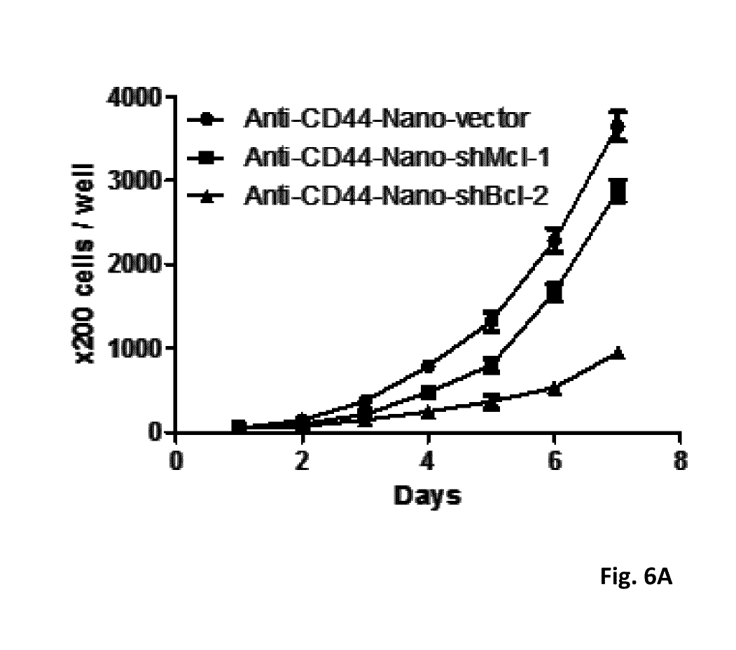

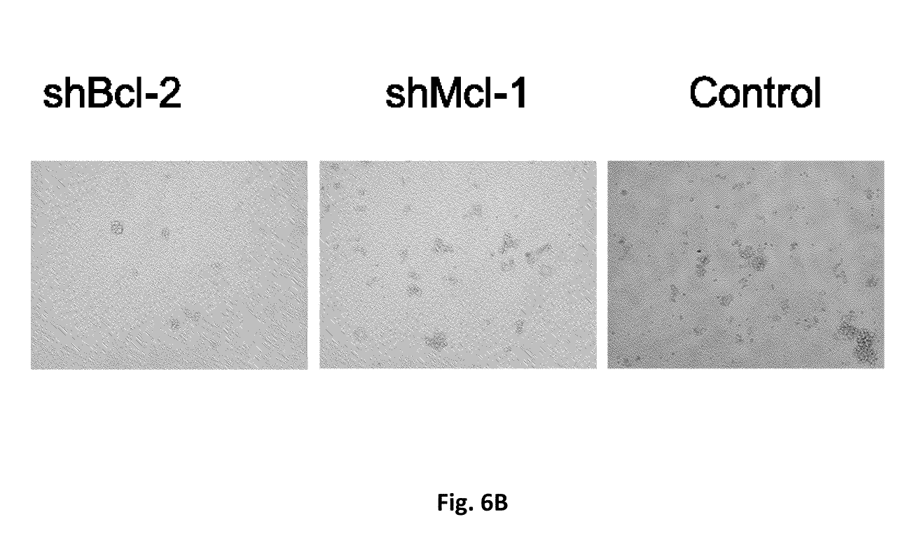

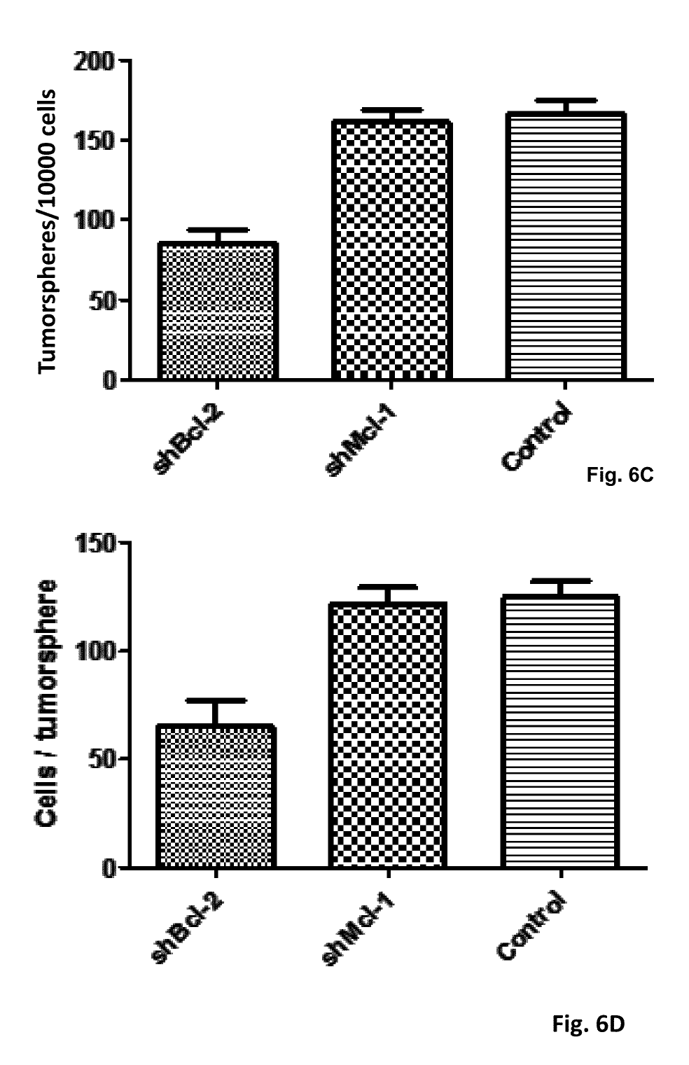

It has been found that anti-CD44-nanovector delivered Bcl-2 shRNA (e.g., Seq ID Nos. 1-2) inhibited cancer cells growth, tumorsphere formation in vitro and tumor initiation in vivo. Cell growth and tumorsphere assays have been conducted. Bcl-2 is a critical oncogene which protects cancer cells and may play a role in cancer stem cells survival by blocking apoptosis. We have evaluated the effect of Bcl-2 shRNA delivered by the anti-CD44-nanovectors on cancer cell growth. The Bcl-2 shRNA in expression plasmid under U6 promoter, pshBcl-2 (e.g., Seq ID Nos. 1-2), has been validated to be effective in knocking down >90% of the target gene expression. MiaPaCa-2 cells were treated with anti-CD44-nanovector-pshBcl-2 in vitro as described above. As shown in FIG. 6A, Bcl-2 shRNA delivered by anti-CD44-nanovector significantly inhibited tumor cell growth, whereas Mcl-1 shRNA or double-strand hybrid siRNA (hsRNA) (e.g., Seq ID Nos. 3-4) showed minimal effects, suggesting Bcl-2 is a valid target for the MiaPaCa-2 cells.

We also evaluated the effect of Bcl-2 shRNA delivered by the anti-CD44-nanovectors on pancreatic cancer tumorsphere formation and growth. MiaPaCa-2 cells were treated with anti-CD44-nanovector-pshBcl-2 in vitro as described above. 48 hours later, cells were trypsinized and 10,000 cells were seeded per well in a low affinity six-well plates for tumorsphere formation. 2 ml DMEM were added to each well supplemented with EGF and FGF growth factors. The tumorspheres were counted under microscopy. All tumorspheres were collected and digested with 0.25% trypsin for five minutes, single cell suspension was made for cell counting, and the numbers of cells per tumorsphere were calculated. As shown in FIGS. 6B-6C, Bcl-2 shRNA delivered by anti-CD44-nanovector significantly inhibited tumorsphere formation, and the tumorspheres were significantly smaller than vector control (see FIG. 6D) (p<0.01, n=3).

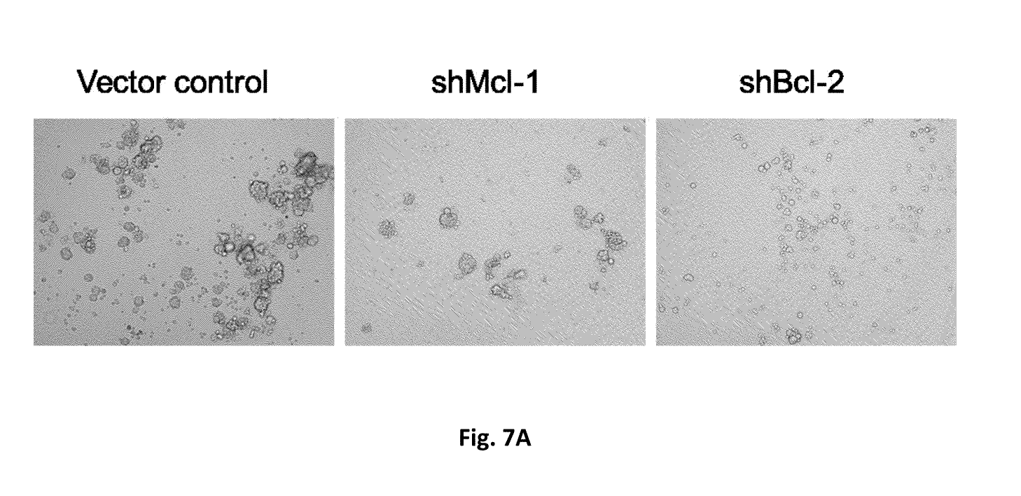

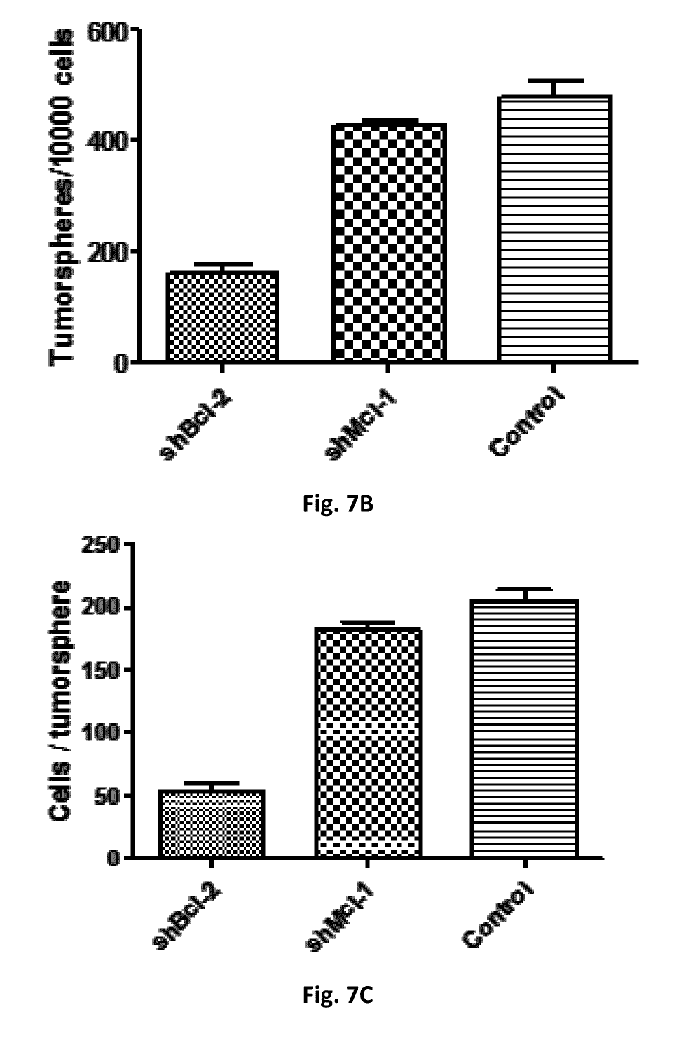

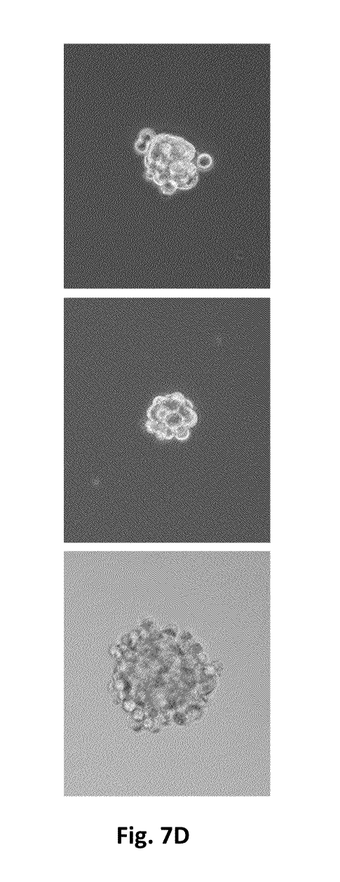

Anti-CD44-nanovector delivered Bcl-2 shRNA inhibited human breast cancer cells growth. FIGS. 7A-7C shows the same results in human breast cancer MDA-MB-231 cells. Other cancer cells were also tested and showed similar results. Also, FIG. 7D shows images of tumorspheres for reference.

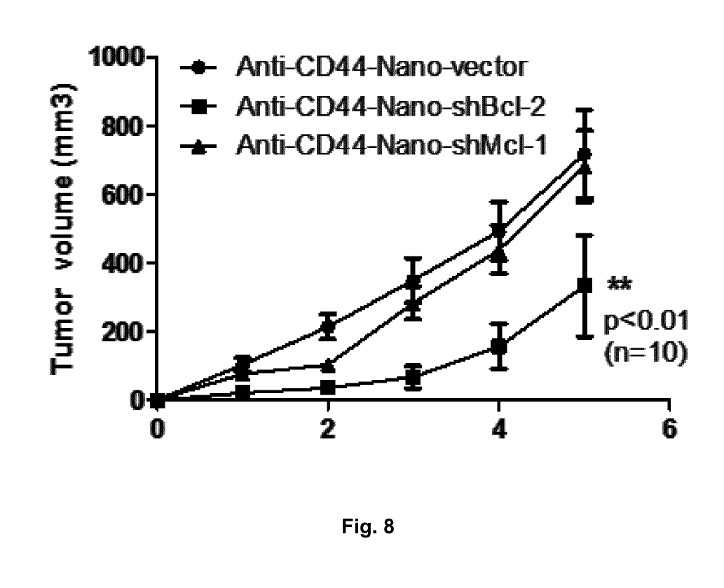

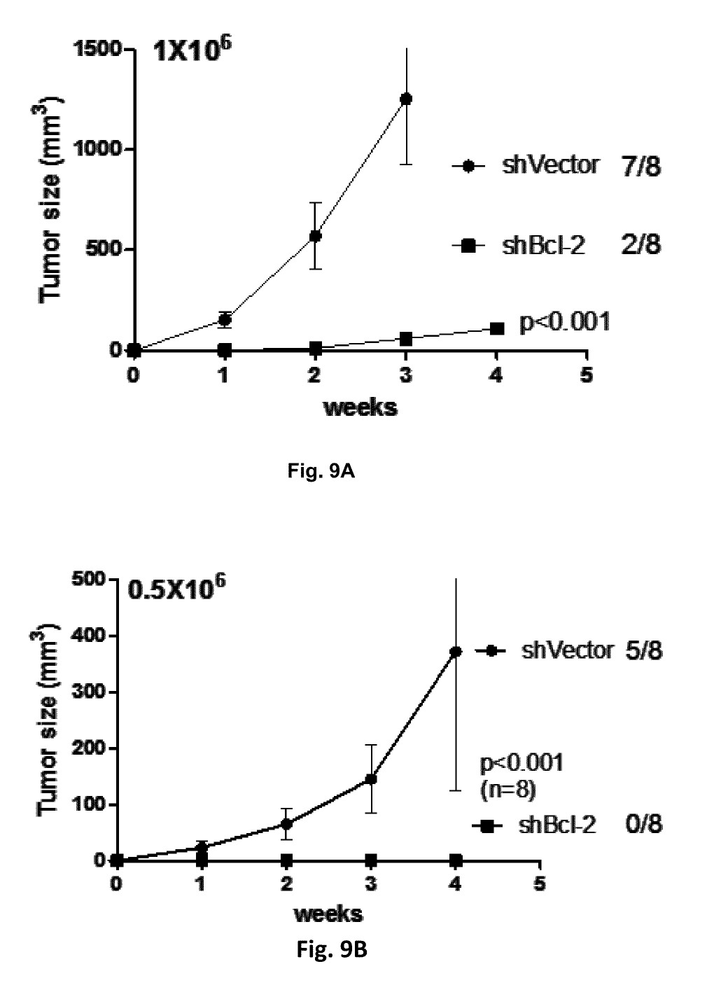

Additionally, tumor initiation assays were conducted. We have evaluated the effect of Bcl-2 shRNA delivered by the anti-CD44-nanovectors on tumor initiation in vivo in nude mice. MiaPaCa-2 cells were treated with anti-CD44-nanovector-pshBcl-2 in vitro as described above. 48 hours later, cells were trypsinized and injected subcutaneously in athymic nude mice, 1×10⁶ cells per site. The tumor formation was observed and tumor sizes were measured. As shown in FIG. 8, Bcl-2 shRNA delivered by anti-CD44-nanovector significantly inhibited tumor formation and growth, p<0.01 versus vector control, n=10. FIGS. 9A-9B show the anti-CD44-nanovector delivered Bcl-2 shRNA also inhibited tumor initiation and growth of human breast cancer MDA-MB-231 in vivo in nude mice. More significantly, in mice injected with 0.5×10⁶ cells, anti-CD44-nanovector-pshBcl-2 treated cells completely inhibited tumor initiation (see FIG. 9B).

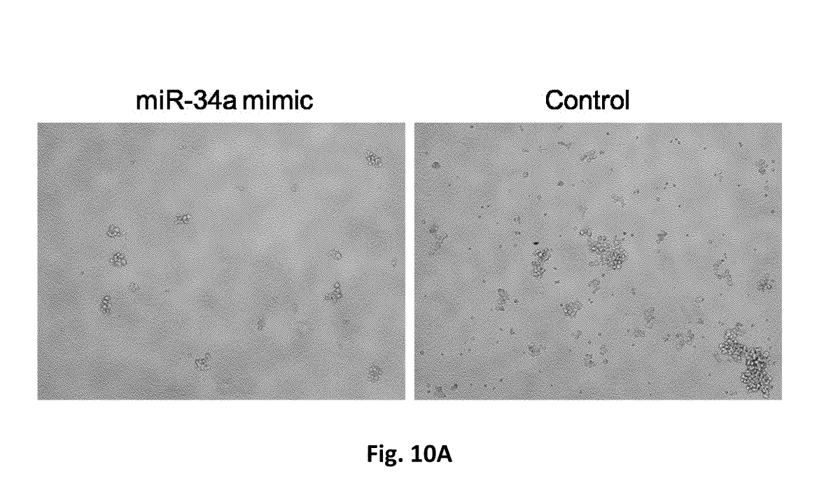

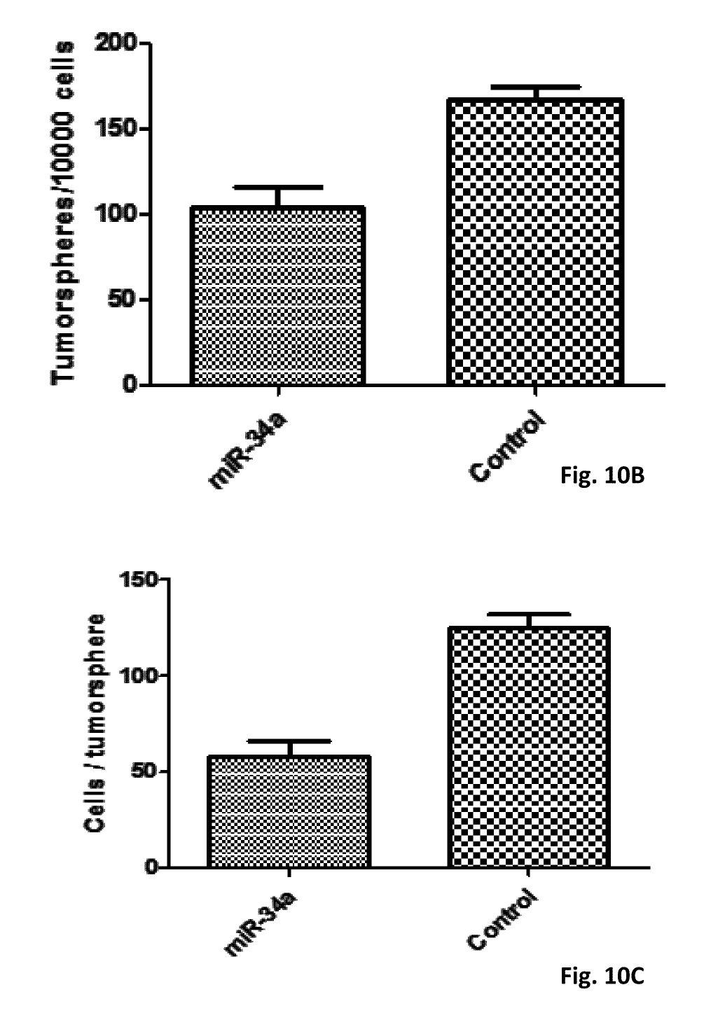

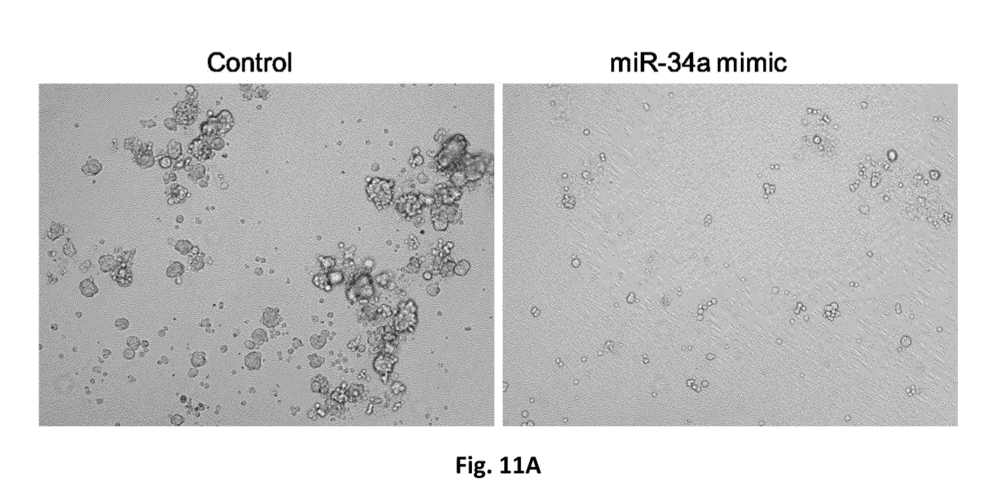

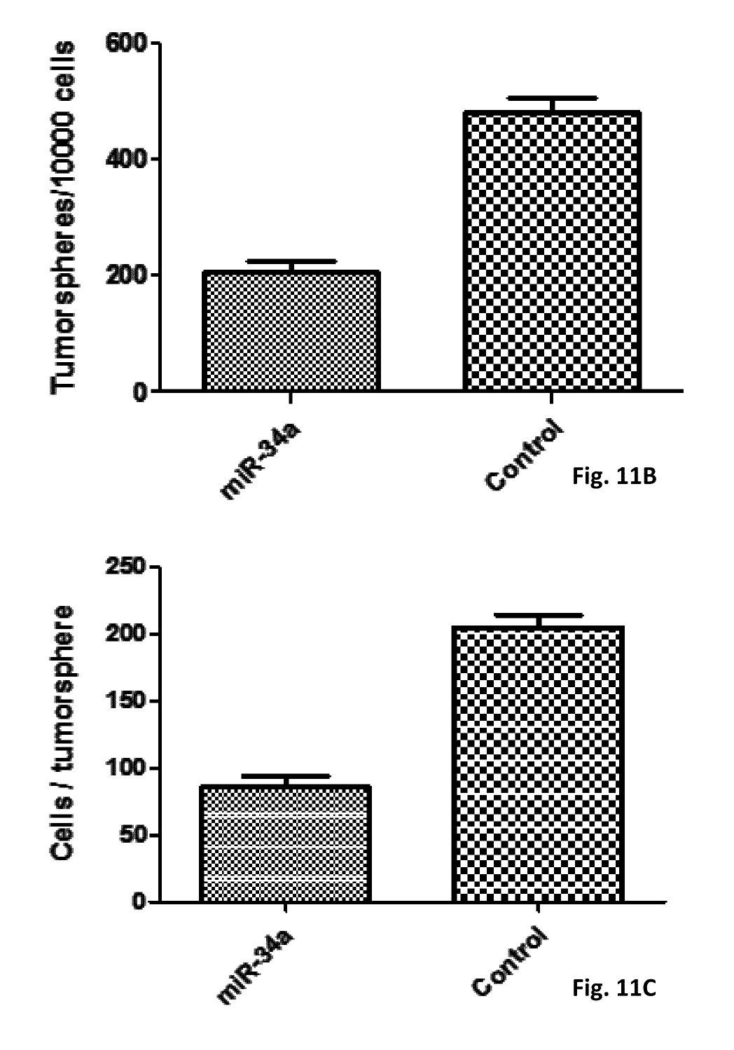

It was found that anti-CD44-nanovector delivered miRNA miR-34a mimic (e.g., Seq ID Nos. 5-6) inhibited cancer cells growth, tumorsphere formation in vitro and tumor initiation in vivo. Accordingly, cell growth and tumorsphere assays were conducted. The microRNA miR-34 is a p53 target and is reported to be a potential tumor suppressor. The human pancreatic cancer MiaPaCa-2 cells and human breast cancer MDA-MB-231 cells have mutant p53 and we found they have lost miR34 expression. We tested the effects of miR-34 restoration by the anti-CD44-nanovector delivered miR-34 mimics. Methods are the same as above. miR-34a mimic delivered by anti-CD44-nanovector significantly inhibited tumor cell growth of the two cell lines (data not shown). As shown in FIGS. 10A-10C and 11A-11C, miR-34a mimic delivered by anti-CD44-nanovector significantly inhibited tumorsphere formation, and the tumorspheres were smaller than nonspecific control mimic (p<0.01, n=3). Other cancer cell lines were also tested and showed similar results.

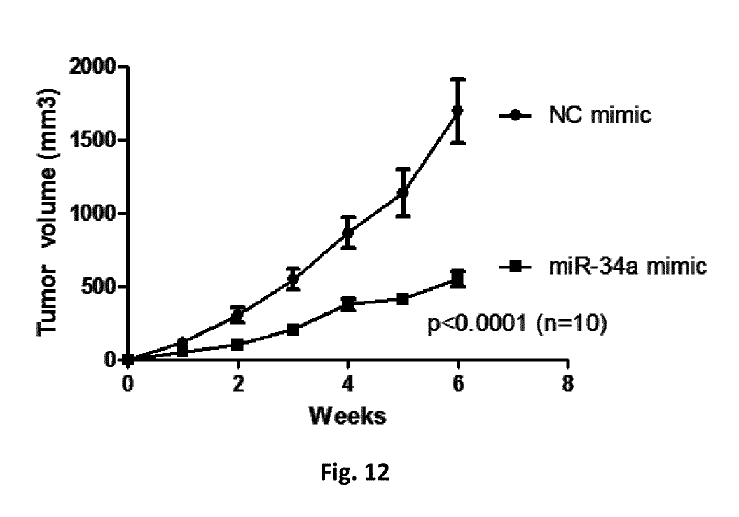

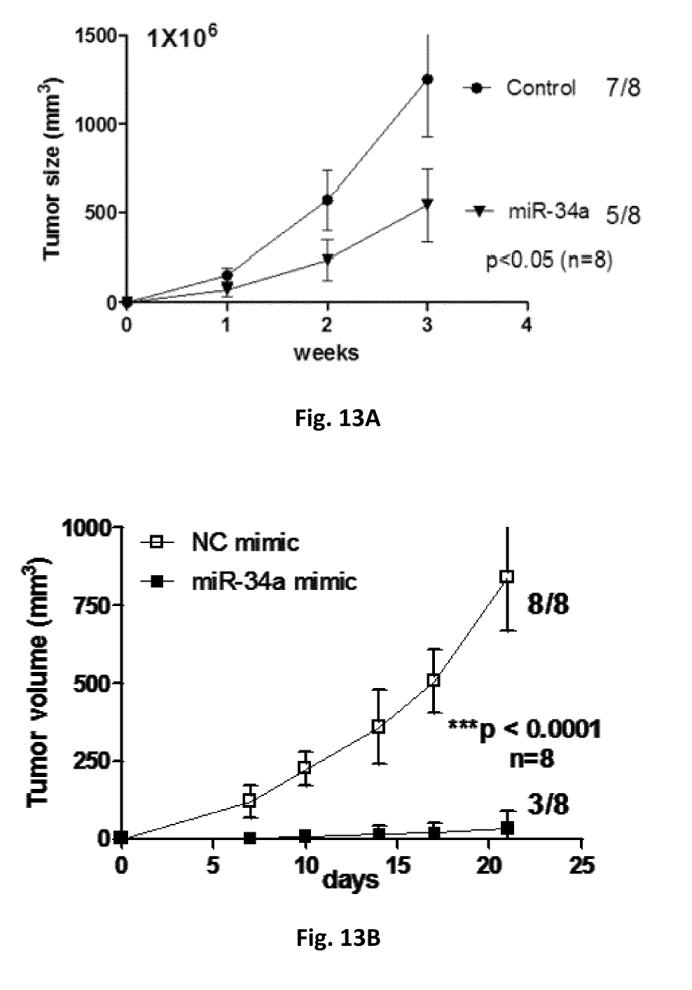

Tumor initiation assays were also conducted. We have also carried out tumor initiation studies to evaluate whether miR-34 delivered by the anti-CD44-nanovectors will inhibit tumor initiation in vivo in nude mice. Cancer cells transfected with miR-34a mimic or negative control (NC) mimic were inoculated subcutaneously in nude mice, and the tumor initiation was monitored. Methods are the same as above. As shown in FIGS. 12 and 13A-13B, miR-34a mimic delivered by anti-CD44-nanovector significantly inhibited tumor initiation, and the tumors were also significantly smaller, p<0.01 and p<0.0001, versus nonspecific control mimic (NC mimic), n=8-10. Our data with miR-34 mimic are consistent with the data from lentiviral miR34. Since lentivirus may not be safe for human therapy and lacks tumor specificity, our anti-CD44-nanovectors disclosed in this patent will provide significant and practical advantage as a safe and tumor-targeted therapy to deliver miR-34 to tumor stem cells. Our data demonstrate that, besides plasmid DNA and siRNA, microRNA can also be delivered by the nanovectors.

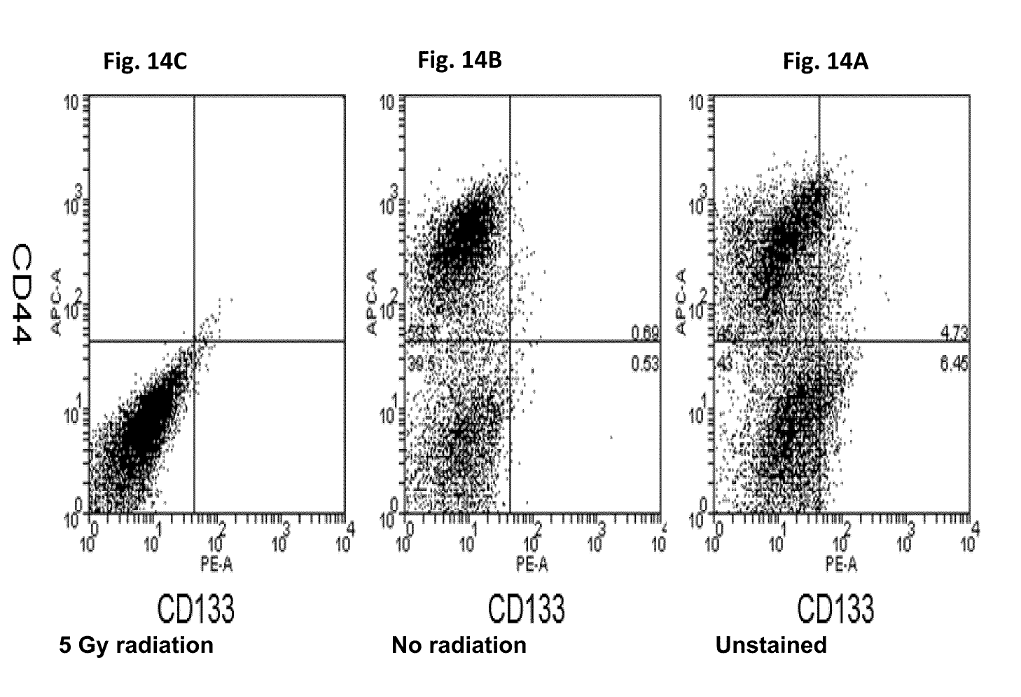

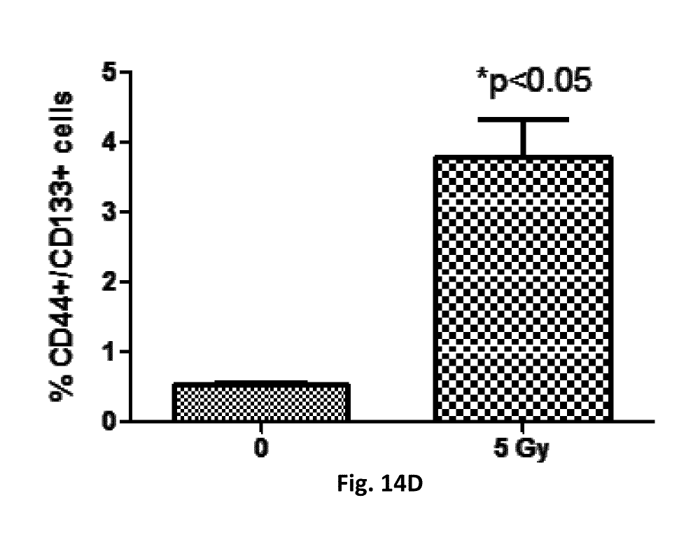

It has been found that radiation increases CD44+/CD133+ tumor initiating cells (TIC) or cancer stem cells (CSC). To evaluate the effects of x-ray radiation on the CD44 and CD133 makers and on the cancer stem cells population, human pancreatic cancer MiaPaCa-2 cells were radiated at 5 Gy. 24 hours later, the cells were collected and stained with APC-anti-CD44 and PE-anti-CD133, then analyzed by flow cytometry. As shown in FIGS. 14A-14D, 5 Gy x-ray radiation significantly increased CD44+/CD133+ tumor initiating cells (TIC) or cancer stem cells (CSC), from 0.53±0.04% (non-radiated control) to 3.78±0.55% (5 Gy), a 7.2-fold increase (p<0.05, n=2). Similar results were observed in cells after chemotherapy. This is consistent with literature that cancer stem cells are resistant to chemo/radiotherapy, and targeting the CD44+/CD133+ tumor initiating cells (TIC) or cancer stem cells (CSC) by our anti-CD44-nanovectors provide a more potent and specific molecular therapy for cancer stem cells and tumor initiating cells.

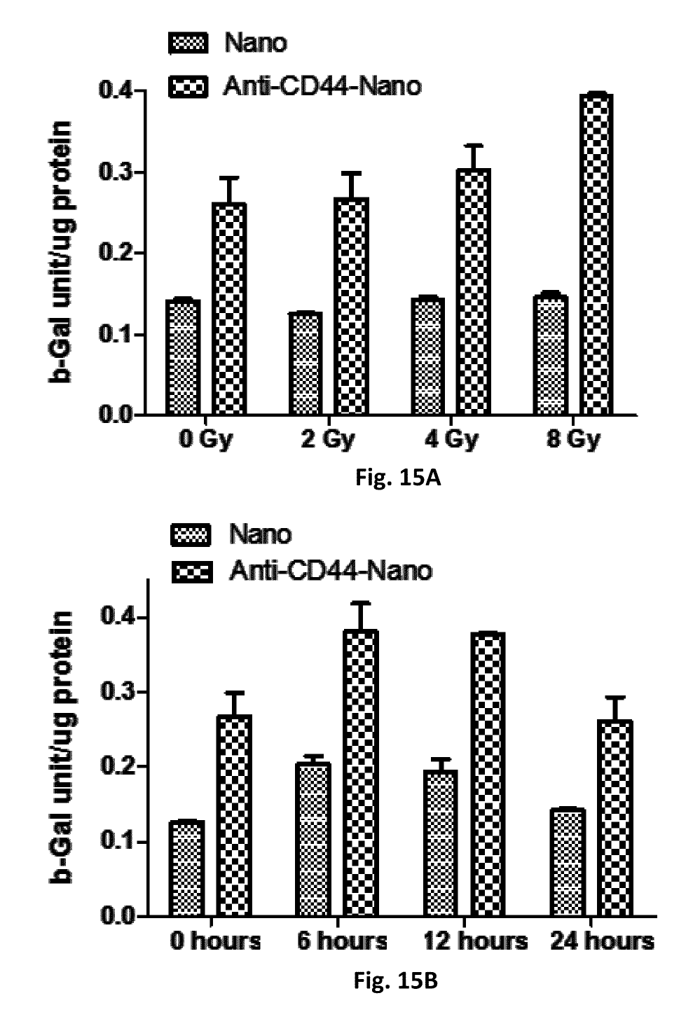

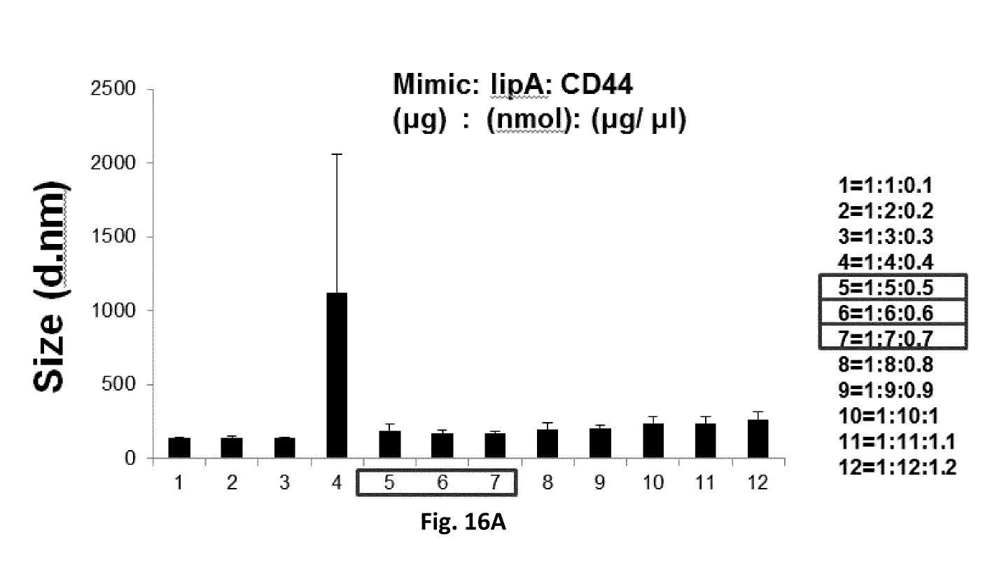

It has been found that radiation increases transfection efficiency of anti-CD44-nanovectors. To evaluate the effects of x-ray radiation on the transfection efficiency of anti-CD44-nanovectors, human pancreatic cancer MiaPaCa-2 cells in 24-well plates were first radiated at 2, 4, and 8 Gy. After the indicated time, the cells were transfected by anti-CD44-nanovector-pCMVb, as described herein. 48 hours later, the cells were collected and lysed for quantitative β-gal enzyme assay. As shown in FIG. 15A, radiation dose-dependently increased transfection efficiency of anti-CD44-nanovectors. Human pancreatic cancer MiaPaCa-2 cells in 24-well plates were radiated at 0, 2, 4, and 8 Gy. After six hours, the cells were transfected by anti-CD44-nanovector-pCMVb. 48 hours later, the cells were collected and lysed for quantitative beta-Gal enzyme assay. 4-8 Gy showed best increase of transfection efficiency of the anti-CD44-nanovectors. FIG. 15B shows that the radiation-mediated increase of transfection efficiency of anti-CD44-nanovectors was dependent on the time between radiation and transfection. The cells were radiated at 2 Gy. After 0, 6, 12, and 24 hours, the cells were transfected by anti-CD44-nanovector-pCMVb. 48 hours later, the cells were collected and lysed for quantitative beta-Gal enzyme assay. Treating cells six hours after radiation shows the best increase of transfection efficiency of the anti-CD44-nanovectors. Treating cells 24 hours after radiation showed no increase by radiation.

Our data demonstrate that radiation can dose- and time-dependently increase transfection efficiency of anti-CD44-nanovectors. This increase is accompanied by the radiation-induced increase of CD44+/CD133+ tumor initiating cells (TIC) or cancer stem cells (CSC) (FIGS. 14A-14D). The data indicate that with optimal conditions, including (1) the optimized nanovector formulation (below), (2) optimal dose and timing of radiation and treatment, and (3) optimal anti-CD44-nanovector dose and schedule, especially repeated systemic delivery in vivo, based on PK and biodistribution, we may be able to achieve the highest possible in vivo delivery efficiency to cancer stem cells, and efficacy in terms of long-term survival.

Optimization of nanovectors in Different Cancer Cell Lines has been conducted. In this example we further explored the ligand-cationic liposome system, preparing a panel of ligand-targeted cationic liposomes to optimize the transfection efficiency to a variety of human and rodent cancer cells.

Cationic liposomes were prepared as follows (Modified from U.S. Pat. No. 6,749,863):

LipA DOTAP/DOPE: 1:1 molar ratio;

LipB DDAB/DOPE: 1:1 molar ratio;

LipC DDAB/DOPE: 1:2 molar ratio;

LipD DOTAP/Chol; 1:1 molar ratio;

LipE DDAB/Chol; 1:1 molar ratio;

LipG DOTAP/DOPE/Chol; 2:1:1 molar ratio; and

LipH DDAB/DOPE/Chol; 2:1:1 molar ratio.

Targeting ligands were prepared. Each of the above formulations mixed with the followings in medium or buffer, then mixed with reporter gene plasmid DNA, oligos, RNA, miRNA, etc., in medium or buffer to form the complex:

(1) anti-CD44 antibody or scFv fragments;

(2) holo-transferrin (Tf); and

(3) anti-transferrin receptor antibody or scFv fragments.

The firefly luciferase gene in plasmid pCMVLuc or E. coli beta-galactosidase gene in plasmid pCMVb, as well as GFP or RFP in plasmids pGFP or pRFP, were used as a reporter gene.

Contents delivered by nanovectors can include the following: plasmid DNA (genes, shRNA, miRNA, etc.); DNA fragment (shRNA/miRNA expression cassettes); RNA; Oligos; DNA/RNA hybrids; siRNA; and miRNA mimics.

Preparation of DNA-liposome complexes was performed.

The various DNA-liposome-transferrin complexes were prepared by the addition of protein ligand to serum-free medium. 5-15 minutes later, cationic liposome (LipA, LipB, LipC, LipD, LipE, LipG, and LipH) was added and mixed. After 5-15 minutes incubation at room temperature with frequent rocking, an equal amount of medium containing reporter gene plasmid DNA was added and mixed, and incubated 15-30 minutes at room temperature with frequent rocking. The DNA/lipid/protein ratios in optimization were in the range of 1/(0.1-50)/(0.1-100) μg/nmol/μg.

Preparation of miRNA mimic anti-CD44-nanovectors was performed. The various ratios of anti-CD44-nanovectors were prepared by the addition of CD44 antibody and cationic liposome (LipA) to distilled water and mixed. After 15 minutes incubation at room temperature with frequent rocking, an equal amount of distilled water containing miRNA mimic was added and mixed, and incubated 15-30 minutes at room temperature with frequent rocking. The miRNA mimic/lipid/antibody ratios in optimization were in the range of 1/(1-12)/(0.1-1.2) μg/nmol/μg.

Optimization was performed on the following cell lines: Human squamous cell carcinoma of head and neck: HN17B, HN22a, HN-38, and SCC-25; Human breast cancer: MDA-MB-231, MDA-MB-435, MDA-MB-453, and MCF-7; Human prostate cancer: DU145, LNCaP, CL-1, PC-3, and VCaP; Human ovary cancer: SKOV-3; Human pancreatic cancer: PANC-1, MiaPaCa-2, and BXPC-3; Human colon cancer: SW480 and LS 174T; Human glioblastoma: U-87; Human cervical cancer: HTB-34; Human lung cancer: A549; Human gastric cancer: Hs 746T and KATO-III; Human normal breast epithelial: Hs578Bst; Human endothelial: HUVEC; and Mouse melanoma: B16/F10.

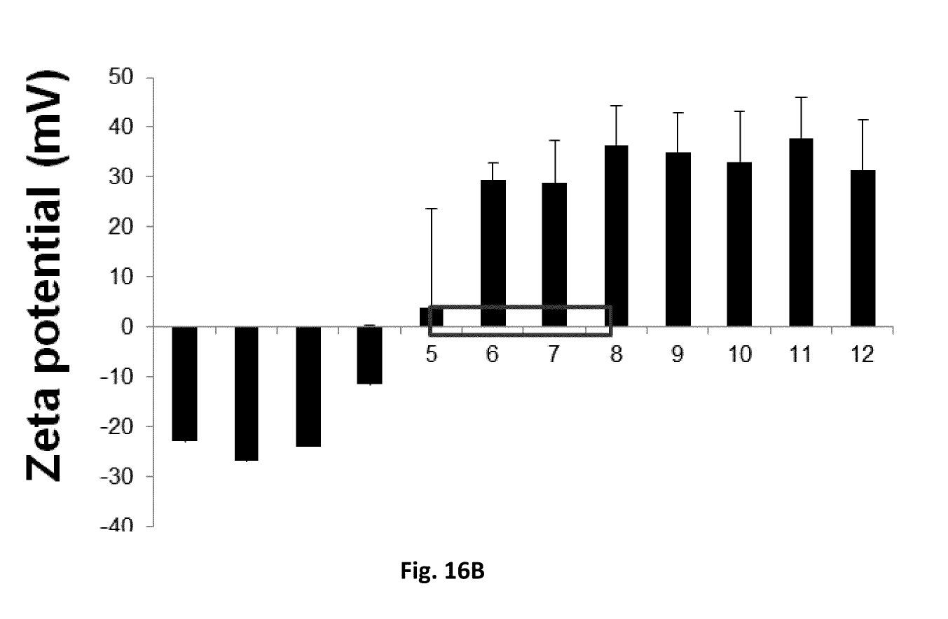

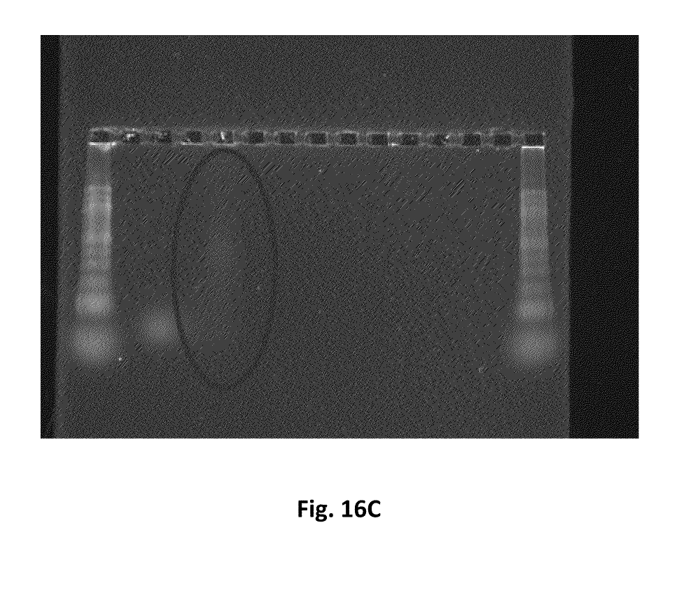

Characterization of anti-CD44-nanovector-miRNA-mimic was performed. Analysis of the size and charge on Anti-CD44-nanovectors-miRNA mimic were performed by examining their sizes and zeta potentials with a ZetaSizer 3000Hsa (Malvern Instruments, MA, USA). The system was calibrated with a −69.8 mV standard (DTS 50/50 standard, Malvern Instruments) as recommended by the manufacturer. Experimental samples (1 ml) were measured 30 times for one minute. The diameters of Anti-CD44-nanovector-miRNA mimic were ranging from 128.4 to 211.6 nm as showed in FIGS. 16A-16C, as measured by Zetasizer. The surface charge of nanovectors was determined by measuring the zeta potential. As shown in FIG. 16B, at pH 7-8, the nanovectors have zeta potentials from −24.1 to +40.9 mV, respectively.

It has been shown that anti-CD44-nanovector-miRNA-mimic mediates miR34a delivery into cultures cells and down-regulates the target protein expression. In this example, we further optimized anti-CD44-nanovectors-miRNA to improve the in vitro transfection efficiency. PC-3 cells were seeded in six-well plates and incubated for 24 hours prior to transfection, by which time the cells were 70% confluent. Cells were washed once with DMEM medium without serum and antibiotics. For a typical preparation with optimized in vitro formulation, 1.25 μl of anti-CD44 antibody (H4C4, 0.5 mg/ml), and 5 μl of LipA (2 nM total lipids) plus 200 μl of DMEM medium were mixed in a polypropylene tube and incubated for 15 minutes at room temperature with frequently rocking 1 μg of miRNA miR34a mimics in 200 μl DMEM medium was added to the anti-CD44-liposome complex tube, mixed immediately and thoroughly, and incubated for 15 minutes at room temperature with frequently rocking. The final miRNA:Lipid:anti-CD44 ratio was 1:5:0.5 (μg/nmol/μg). 400 μl of transfection solution containing Anti-CD44-liposome-miRNA (either miR34a or NC control mimic) were added to each well. After five hours of transfection at 37° C., 1 ml of DMEM containing 20% FBS was added to each well.

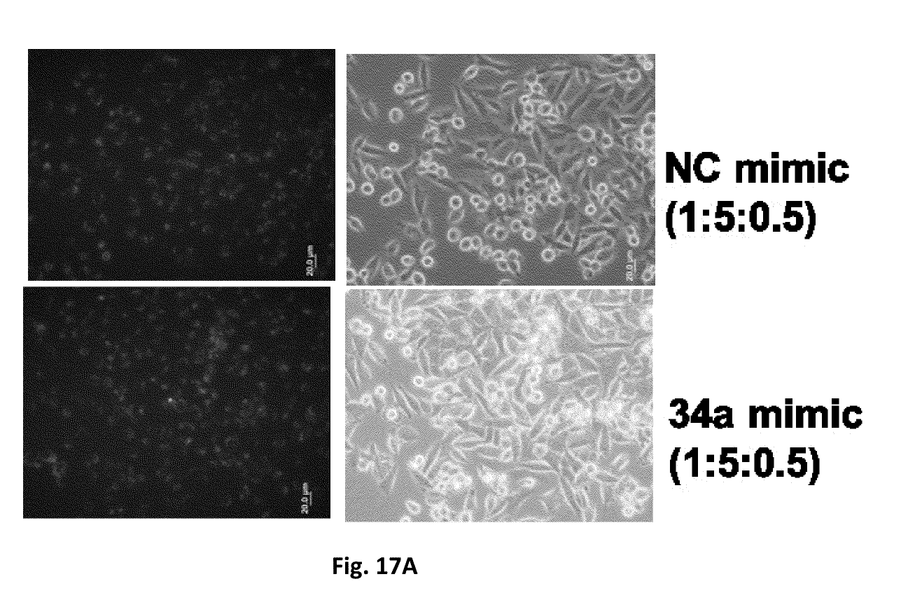

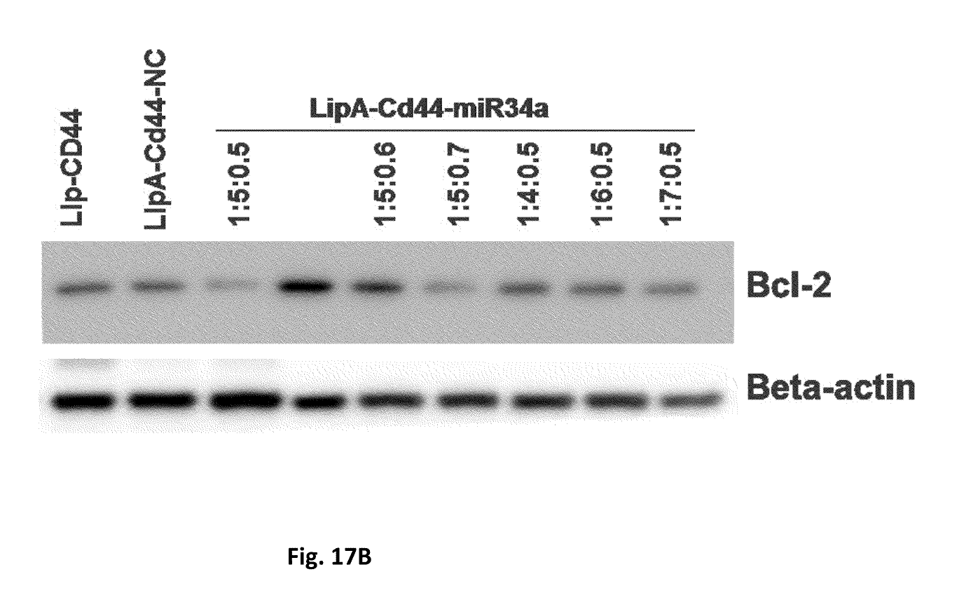

As shown in FIG. 17A, anti-CD44-nanovectors efficiently delivered miR34a mimic to human prostate cancer PC-3 cells, with a transfection efficiency of 90%. FIG. 17B shows Bcl-2, a target protein of miR34a, was significantly down-regulated with a ratio of miRNA/LipA/anti-CD44 antibody ranging from 1:5:0.5 to 1:5:0.7. When we put these data together, we choose the ratio of miRNA/LipA/anti-CD44 antibody at 1:5:0.5 (ug/nmol/ug) as the optimal ratio for in vivo studies.

In vivo optimization of anti-CD44-nanovectors targeting cancer stem cells has been conducted. In this example we further optimized the anti-CD44-nanovectors to improve the in vivo transfection efficiency to cancer stem cells.

Preparation of anti-CD44-nanovectors containing FITC-siRNA (anti-CD44-Nano-siRNA) includes:

(1) LipA 900 μl and anti-CD44 mAb H4C4 (0.5 mg/ml) 180 μl and mix well, incubate for 10 minutes at room temperature;

(2) FITC-siRNA 250 μl (1 mg/ml), add H2O 20 μl and mix well, incubate for 10 minutes;

(3) Add the FITC-siRNA to LipA/anti-CD44 and mix well, incubate for 20 minutes; and

(4) Add 150 μl 50% glucose.

Preparation of anti-CD44-nanovectors containing AcGFP plasmid (anti-CD44-Nano-pGFP) includes:

(1) LipA 500 μl and anti-CD44 mAb H4C4 (0.5 mg/ml) 100 μl and mix well, incubate for 10 minutes at room temperature;

(2) pGFP 100 μl (1 mg/ml), add H2O 200 μl and mix well, incubate for 10 minutes;

(3) Add the pGFP to LipA/anti-CD44 and mix well, incubate for 20 minutes; and

(4) Add 100 μl 50% glucose.

In vivo studies were conducted. Anti-CD44-nanovectors containing FITC-siRNA (anti-CD44-nano-siRNA) and anti-CD44-nanovectors containing AcGFP plasmid (anti-CD44-nano-pGFP) were injected i.v. into nude mice bearing MiaPaCa-2 xenografts, as indicated below for dose schedule, and the tumors were collected as indicated. The tumors were treated with collagenase to obtain single cells, stained with APC-anti-CD44 and PE-anti-CD133, and then analyzed by flow cytometry. Table 1 outlines the in vivo studies.

TABLE 1

| Regimen | Day 1 | Day 2 | Day 3 |
|---|---|---|---|
| anti-CD44- | 250 μg/mouse | 250 μg/mouse | Collect tumors |
| Nano-siRNA | i.v. | i.v. | |
| anti-CD44- | 100 μg/mouse | 100 μg/mouse | Collect tumors |
| Nano-pGFP | i.v.X2 | i.v. | |

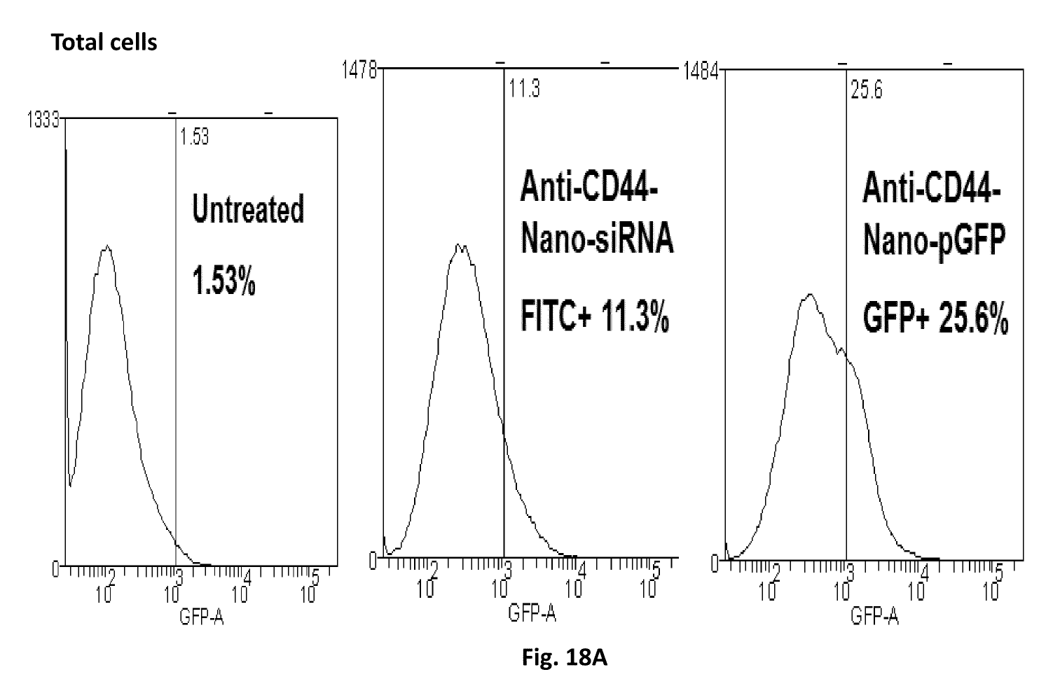

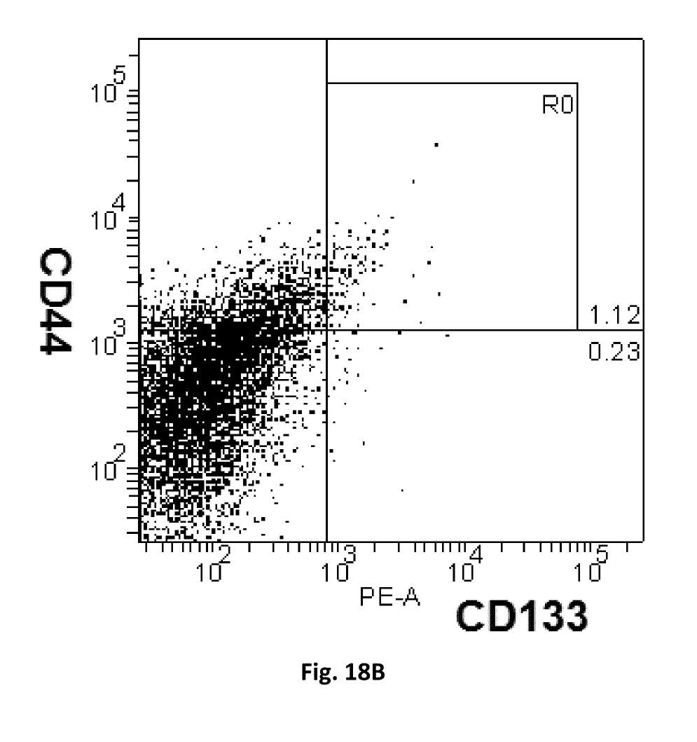

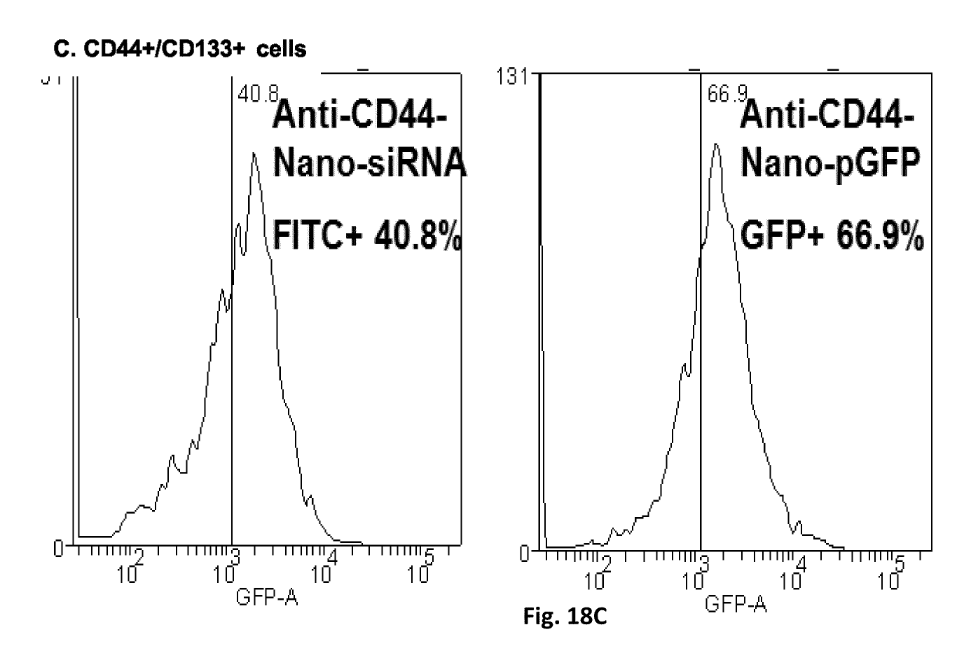

As shown in FIGS. 18A-18C, with the optimized formulation of nanovectors and optimized dose schedule, anti-CD44-nanovector can target FITC-siRNA to 40.8% of CD44+/CD133+ cancer stem cells, and target GFP plasmid to 66.9% of CD44+/CD133+ cancer stem cells (FIG. 18C), up from 11.3% and 25.6% in total cells, respectively (FIG. 18A). Nanovectors with these levels of delivery efficiency are so far the most efficient gene/siRNA delivery system for cancer stem cells ever reported.

In vivo tumor targeted delivery of miRNA by anti-CD44-nanovectors and transferrin-nanovectors in animal tumor models was studied. In this example we examined the anti-CD44-nanovectors and transferrin-nanovectors for in vivo tumor targeted delivery of miRNA therapeutics in xenograft tumor models. Preparation of transferrin-nanovectors containing miR34a/Vector plasmid (Tf-Nano-miR34a/Vector plasmid):

(1) LipA (2 nM) 150 μl and Tf (5 mg/ml) 75 μl and mix well in 9 μl H₂O, incubate for 10 minutes at room temperature;

(2) miR34a plasmid 30 μl (1 mg/ml), add into 6 μl H₂O and mix well, incubate for 10 minutes;

(3) Add the miR34a plasmid to Tf/LipA complex and mix well, incubate for 10 minutes; and

(4) Add 30 μl of 50% glucose.

Preparation of transferrin-nanovectors containing either miR34a mimic or negative control mimic (Tf-Nano-miR34a or NC mimic):

(1) LipA(2 nM) 125 μl and Tf (5 mg/ml) 62.5 μl and mix well, incubate for 10 minutes at room temperature;

(2) Add miR34a mimic or NC mimic 87.5 μl (25 μg) into 40 μl H₂O and mix well, incubate for 10 minutes;

(3) Add miR34a or NC mimic to Tf/LipA complex and mix well, incubate for 10 minutes; and

(4) Add 25 μl 50% glucose.

Preparation of anti-CD44-nanovectors containing miR34a/Vector plasmid (anti-CD44-nano-miR34a/Vector plasmid):

(1) LipA 75 μl and anti-CD44 mAb H4C4 (0.5 mg/ml) 30 μl and mix well into 20 μl H₂O, incubate for 15 minutes at room temperature;

(2) miR34a plasmid 30 μl (1 mg/ml), add into H₂O 15 μl and mix well, incubate for 10 minutes;

(3) Add the miR34a plasmid to LipA/anti-CD44 and mix well, incubate for 20 minutes; and

(4) Add 25 μl 50% glucose.

Preparation of anti-CD44-nanovectors containing either miR34a mimic or NC mimic (anti-CD44-nano-miR34a/NC mimic):

(1) LipA 75 μl and anti-CD44 mAb H4C4 (0.5 mg/ml) 30 μl and mix well, incubate for 15 minutes at room temperature;

(2) Add miR34a mimic or NC mimic 87.5 μl (25 μg) into 40 μl H₂O and mix well, incubate for 10 minutes;

(3) Add miR34a mimic or NC mimic to LipA/anti-CD44 and mix well, incubate for 20 minutes; and

(4) Add 25 μl 50% glucose.

In vivo delivery studies were conducted. Accordingly, systemic delivery of miR34a with Tf-nanovectors was conducted in xenograft tumor models. Tf-nanovectors containing miR34a plasmid (Tf-Nano-miR34a P) and Tf-nanovectors containing miR34a mimic (Tf-Nano-miR34aM) were injected i.v. into nude mice bearing prostate cancer (PC-3) or breast cancer (UM2) xenografts as indicated below for dose schedule, and the tumors were collected as indicated. The proteins were extracted with RIPA lysis buffer and the total RNAs were isolated with TRIzol buffer from tumor samples.

| Regimen | Day 1 | Day 2 | Day 3 | Day 4 |
|---|---|---|---|---|
| Tf-Nano- | 30 μg/mouse | 30 μg/mouse | 30 μg/mouse | Collect tumors |
| miR34a P | i.v. | i.v. | i.v. | |
| Tf-Nano- | 25 μg/mouse | 25 μg/mouse | 25 μg/mouse | Collect tumors |
| miR34a M | i.v. | i.v. | i.v. | |

Systemic delivery of miR34a with anti-CD44-nanovectors was conducted in xenograft tumor models. Anti-CD44-nanovectors containing miR34a plasmid (aCD44-nano-miR34aP) and anti-CD44-nanovectors containing miR34a mimic (anti-CD44-nano-miR34aM) were injected i.v. into nude mice bearing prostate cancer (PC-3) or pancreatic cancer (J2) xenografts as indicated below for dose schedule, and the tumors were collected as indicated. The proteins were extracted with RIPA lysis buffer and the total RNAs were isolated with TRIzol buffer from tumor samples.

| Regimen | Day 1 | Day 2 | Day 3 | Day 4 |
|---|---|---|---|---|
| aCD44- | 30 μg/mouse | 30 μg/mouse | 30 μg/mouse i.v. | Collect |
| Nano- | i.v. | i.v. | | tumors |
| miR34a P | | | | |
| aCD44- | 25 μg/mouse | 25 μg/mouse | 25 μg/mouse i.v. | Collect |
| Nano- | i.v. | i.v. | | tumors |
| miR34a M | | | | |

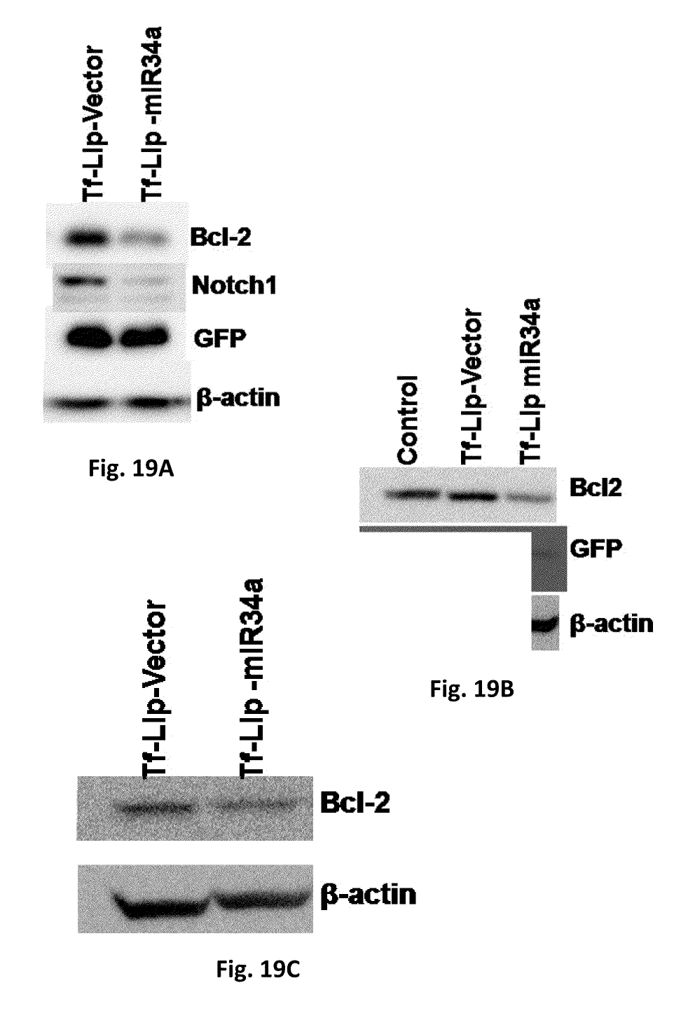

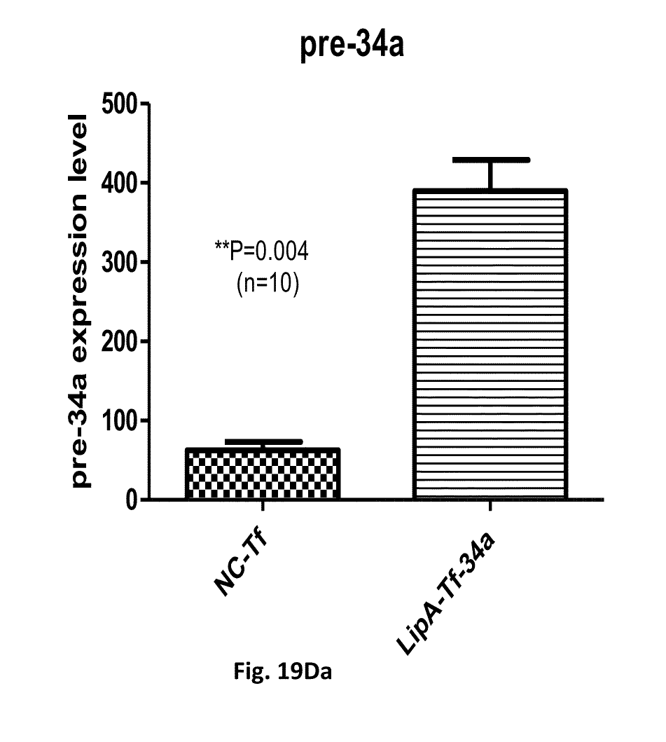

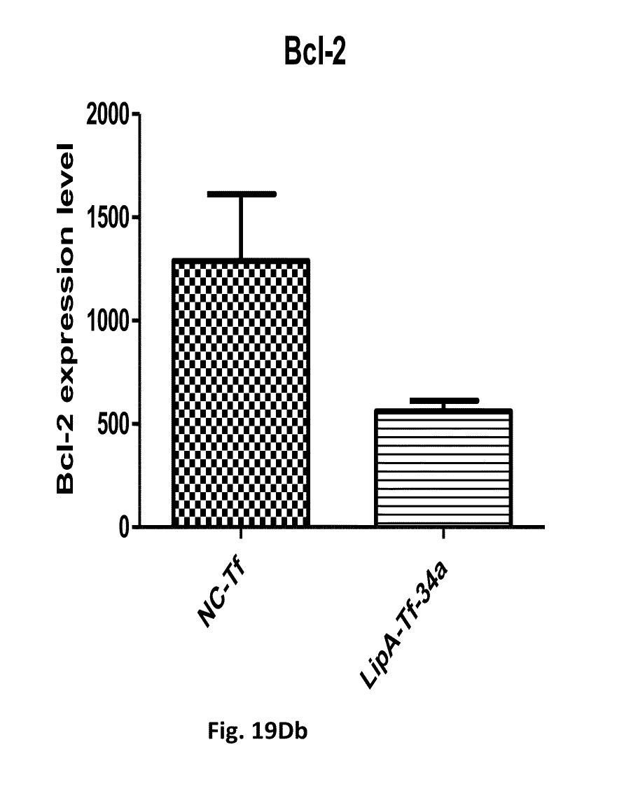

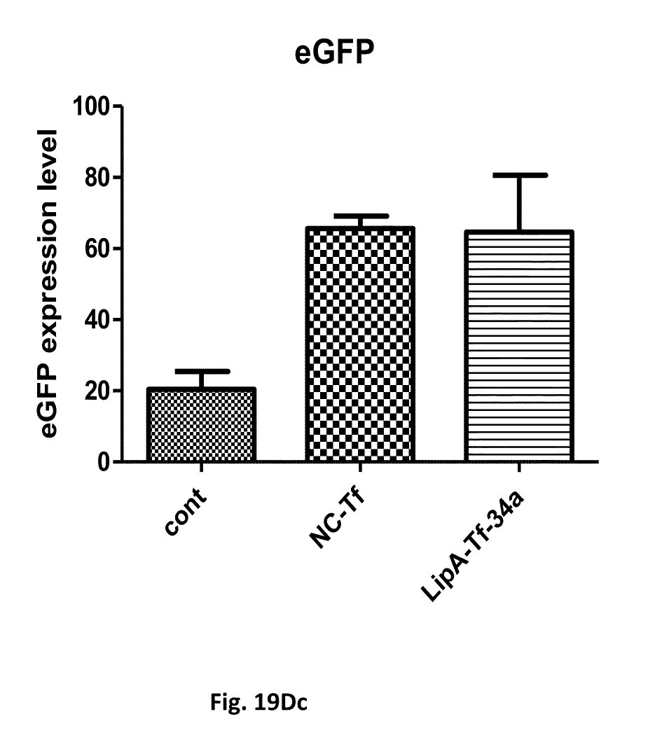

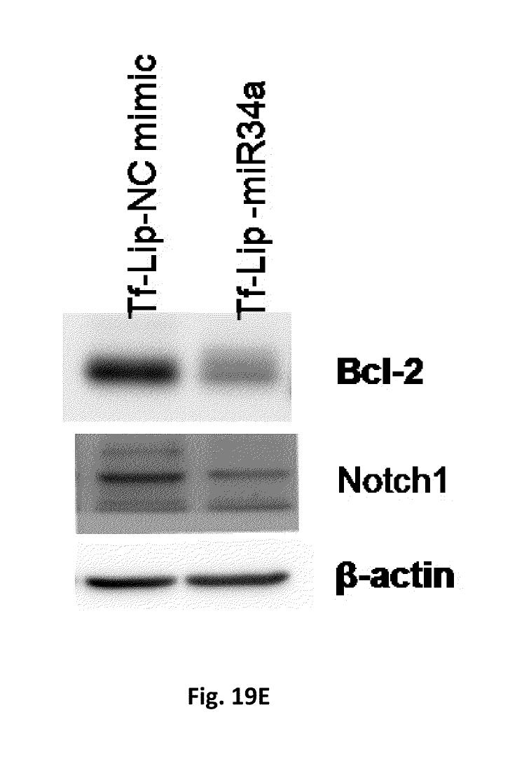

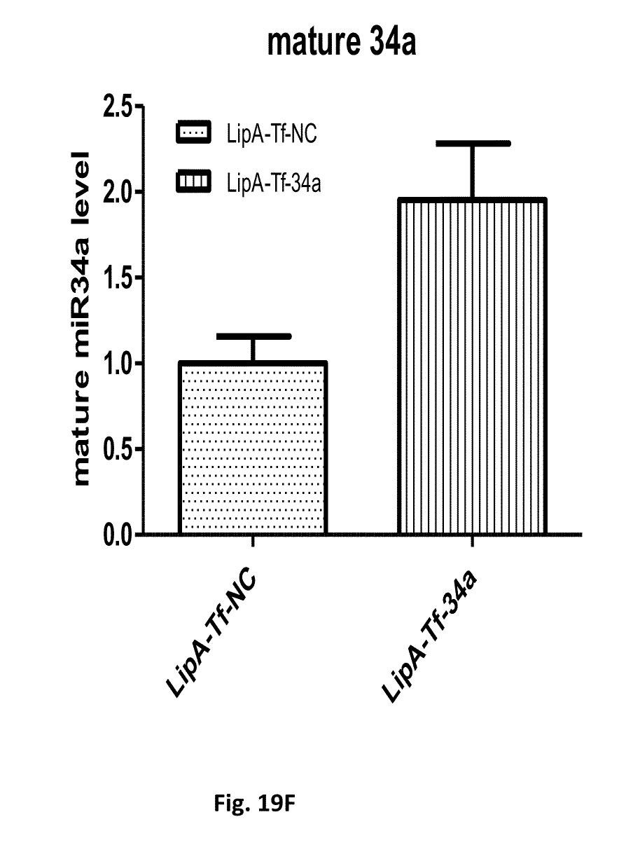

As shown in FIGS. 19A-19C and 19Da-19Dc, systemic miR34a plasmid therapy which delivered with Tf-nanovectors downregulate its targets Bcl-2 and Notch1 expression both in mRNA and protein levels compare with vector treatment in prostate cancer PC-3 xenografts (FIGS. 19A and 19Da-19Dc), breast cancer UM2 xenograft (FIG. 19B), and pancreatic cancer xenograft (FIG. 19C) mice models. To confirm that the miR34a plasmid-delivering vectors were successfully reaching the tumor tissue, we measured the expressions of GFP in xenografts by western blot and qRT-PCR. As shown in FIG. 19A to FIGS. 19Da-19Dc, exogenous GFP expressions can be detected in plasmid treatment group but not for non-treatment control and precursor miR34a increases with miR34a plasmid-delivering nanovector treatment as well. We also observed the Bcl-2 and notch-1 down-regulation of miR34a mimic-delivering nanovectors treated group in prostate cancer xenografts by western blot (FIG. 19E), and meanwhile, using miR34a specific primers, we found significant increases in the expression levels of mature miR34a in xenografts treated with miR34a mimic compared with negative control (FIG. 19F).

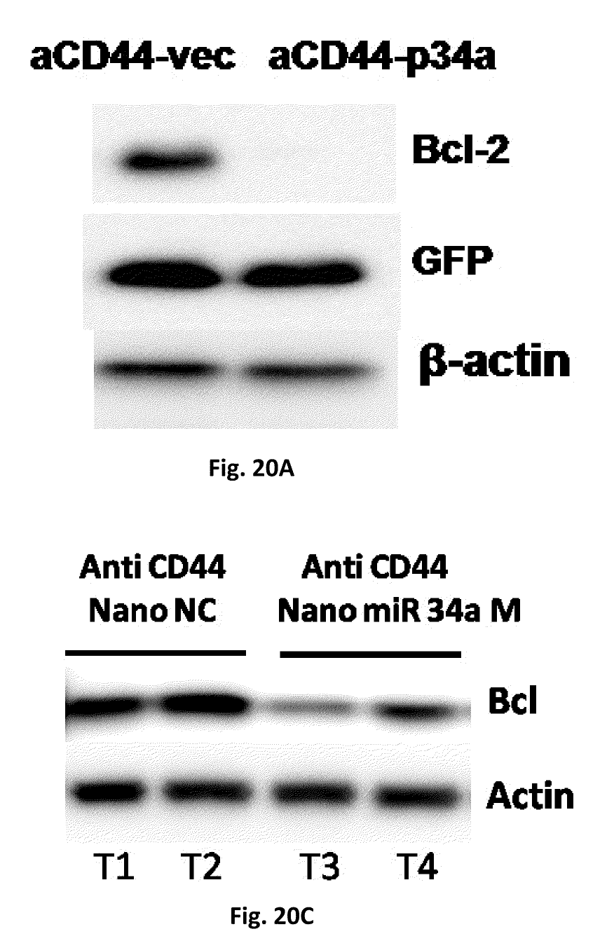

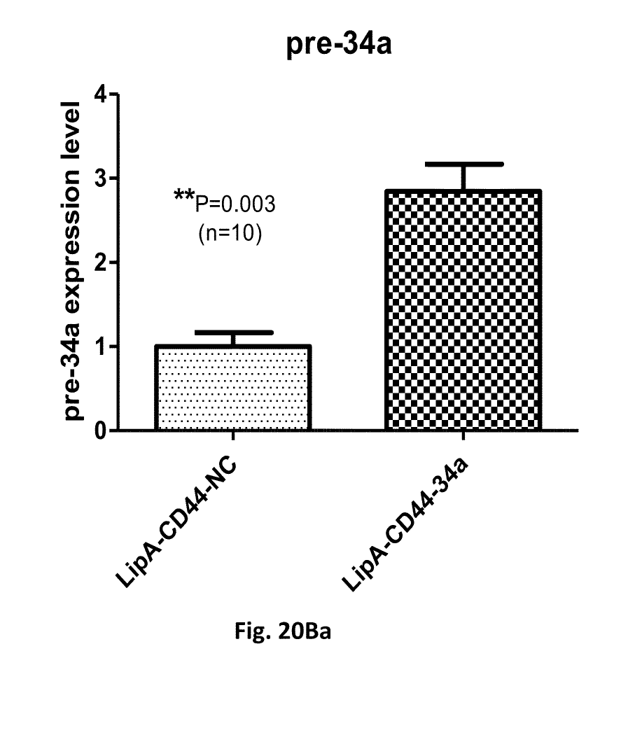

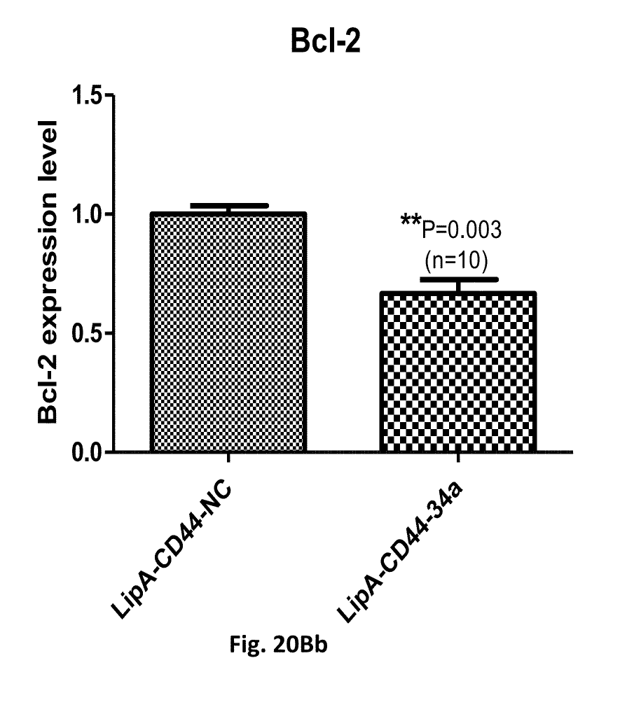

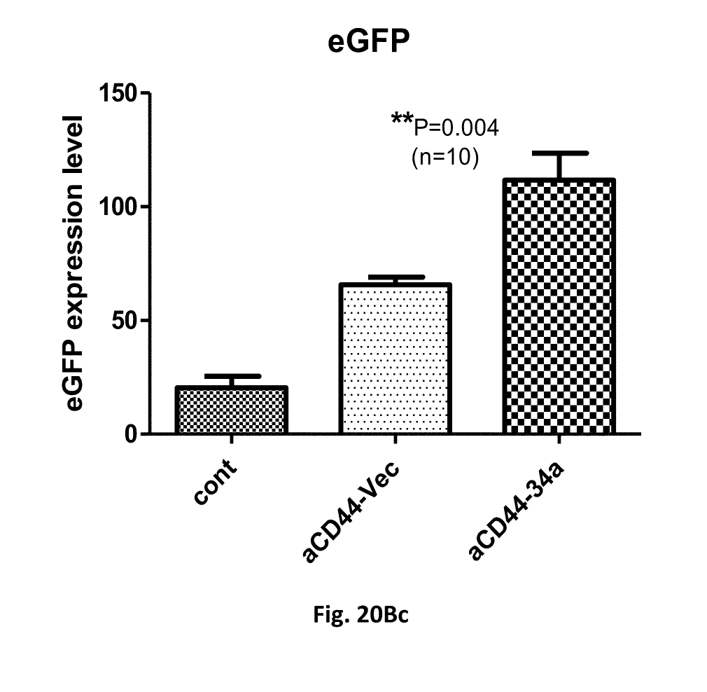

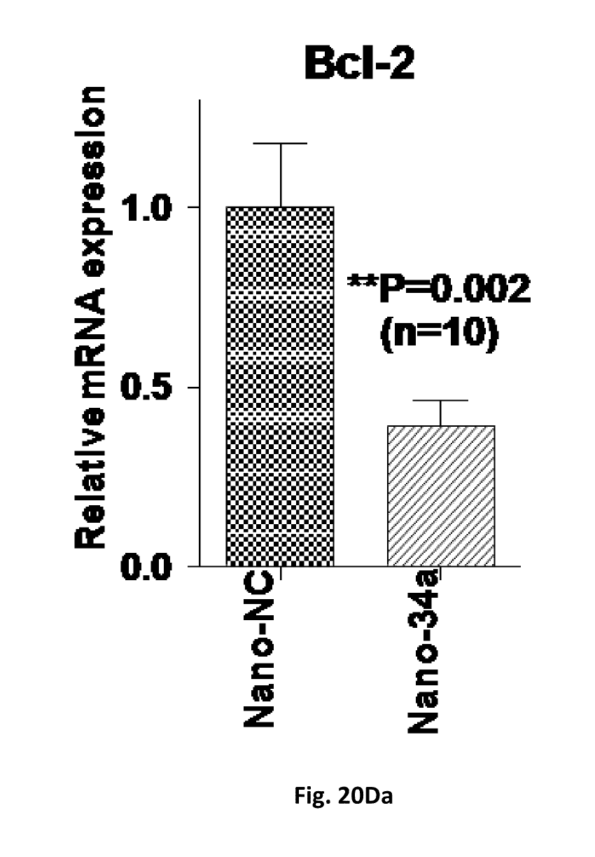

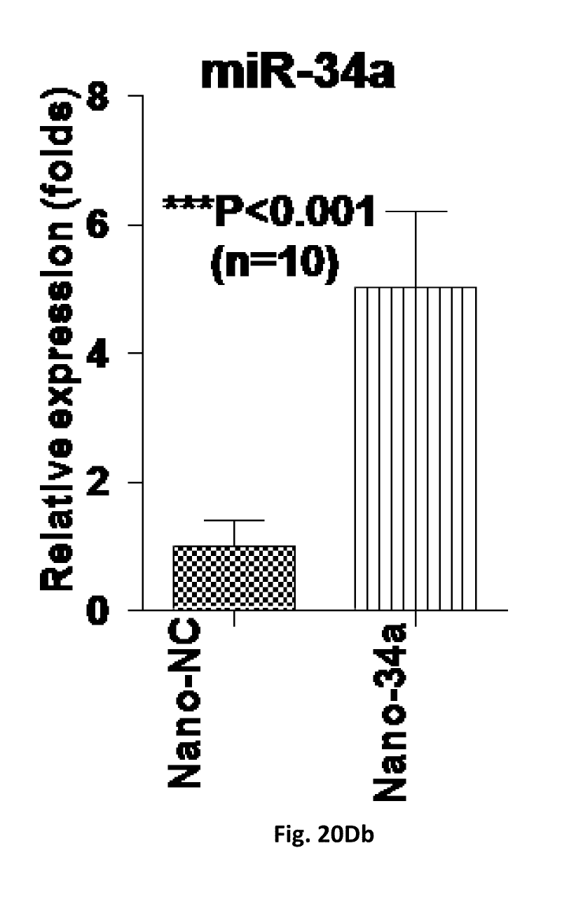

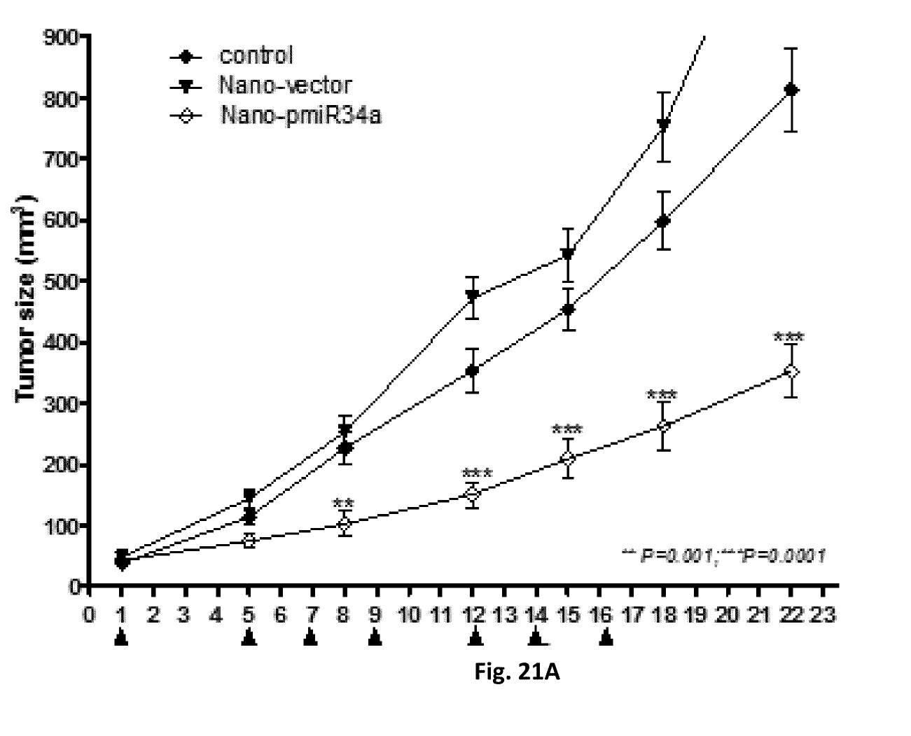

Systemic miR34a plasmid therapy delivered with anti-CD44-nanovectors downregulate its target Bcl-2 expression both in mRNA and protein levels compare with vector treatment in prostate cancer, PC-3 xenografts (FIGS. 20A and 20Ba-20Bc). To confirm that the miR34a plasmid-delivering vectors were successfully reaching the tumor tissue, we measured the expressions of GFP in xenografts by western blot and qRT-PCR. As shown in FIGS. 20A and 20Ba-20Bc, exogenous GFP expressions can be detected in plasmid treatment group but not for non-treatment control group. Precursor miR34a increased with miR34a plasmid-delivering anti-CD44-nanovector treatment as well. We also observed the Bcl-2 down-regulation of miR34a mimic-delivering anti-CD44-nanovectors treated group in prostate cancer xenografts by western blot (FIG. 20C), and meanwhile, using miR34a specific primers, we found significant increases in the expression levels of mature miR34a in miR34a mimic treated xenografts treated compared with negative control (FIGS. 20Da-20Db).

In vivo efficacy of anti-CD44-nanovectors in animal tumor models was studied. In this example we show the in vivo efficacy of the anti-CD44-nanovectors- and transferrin-nanovectors-delivered miRNA therapeutics.

Systemic therapeutic efficacy of miR-34a mimic (Seq ID No. 5) or miR-100 mimic (Seq ID No. 7) delivered with Tf-nanovectors was studied. Athymic nude mice foxn1/nu were purchased from Harlan Laboratories. SUM 159 cells (1×10⁶) were injected subcutaneously into both flank of the nude mice. Four weeks later, the tumors grew to approximately 40-60 mm³ at the injection site. Tf-nanovector-miR100 or Tf-nanovector-NC containing 25 μg of miRNA mimics in 300 μl of 5% glucose were freshly prepared as described above. Nanovectors were intravenously injected 300 μl per mouse via the tail vein, three times a week, for a total of nine injections. The tumor sizes were measured twice a week with a caliper, and calculated as tumor volume=Length×Width²/2.

Tf-nanovectors containing miR34a plasmid (Seq ID No. 6) (Tf-Nano-miR34aP) and Tf-nanovectors containing miR34a mimic (Seq ID No. 5) or miR100 mimic (Seq ID No. 7) (Tf-Nano-miR34a/100 M) were injected i.v. into nude mice bearing prostate cancer (PC-3) or breast cancer (SUM-159) xenografts as described above. The same amount of miRNAs plasmid or mimics were injected for every-other-day (EOD) treatment for three weeks, tumor growth was monitored two/week. All of the tumor size data analysis was by t-test with Graphpad Prism software.

Systemic therapeutic efficacy of miR34a/miR100 delivered with anti-CD44-nanovectors was studied. Anti-CD44-nanovectors containing miR34a plasmid (aCD44-miR34a P (Seq ID No. 6)) and anti-CD44-nanovectors containing miR34a/miR100 mimic (aCD44-Nano-miR34a/100 M) were injected i.v. into nude mice bearing xenografts of human prostate cancer (PC-3) or breast cancer (SUM-159). Nanovectors were freshly prepared as described above, same amount of miRNA plasmid or mimics were used for every-other-day (EOD) treatment for three weeks, tumor growth were monitored two/week. All of the tumor size data analysis was by t-test with Graphpad Prism software.

It was found that systemic miRNA delivery with Tf-nanovectors inhibits the growth of prostate and breast cancer xenografts. PC3 xenografts in nude mice were treated via i.v. injection with either 30 μg of vector or plasmid miR34a Tf-nanovectors for three weeks. Significant tumor growth inhibition was observed when mice were treated with either miR34a plasmid or miR34a/100 mimic Tf-nanovectors, compared with vector and negative controls (FIGS. 21A-21C). Notably, the effects of vector and negative control were essentially identical to non-treated xenografts, indicating that the growth-inhibitory effects were caused by the expression of the target miRNAs. FIGS. 21D and 21E show the unique lamellar structure of Tf-nanovectors by TEM.

It was found that systemic miRNA delivery with anti-CD44-nanovectors inhibits the growth of human prostate and breast cancer xenografts. Prostate cancer PC3 and breast cancer SUM 159 xenografts in nude mice were treated via i.v. injection with either vector or plasmid miR34a anti-CD44-nanovectors for three weeks. Significant tumor growth inhibition was observed when mice were treated with either miR34a plasmid or miR34a/100 mimic aCD44-nanovectors, compared with vector and negative controls (FIGS. 22A-22C). FIGS. 22D and 22E show the unique lamellar structure of anti-CD44-nanovectors by TEM and STEM. The STEM images at the lower panel show an unexpected ordered multi-lamellar nanostructure with a coating of aCD44 antibody on the outer surface, which provides a structural basis for the nanovectors' targeting function.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety: Chang, E. H., Xu, L. and Pirollo, K. F. Targeted liposome gene delivery. International Patent Application No. PCT/US98/24657, filed on Nov. 19, 1998. U.S. Pat. No. 6,749,863, issued on Jun. 15, 2004; Xu, L. and Chang, E. H. Ligand-PEG post-coating stabilized lipoplex and polyplex for targeted gene delivery. U.S. Patent Application No. 60/116,792, filed on Jan. 21, 1999, International Patent Application No. PCT/US00/01346, filed on Jan. 21, 2000; and Xu L., Huang C. C., Alexander W., Tang W. H., and Chang E. H. Antibody fragment-targeted immunoliposomes for systemic gene delivery. U.S. Pat. No. 7,479,276, issued on Jan. 20, 2009.

### Sequences:

(Bcl-2 shRNA):
Seq ID No. 1
TGAATATACACAATCAGGG
(Bcl-2 shRNA):
Seq ID No. 2
ACTTCATCACTATCTCCCG
(Mcl-1 hsRNA (double-strand hybrid siRNA),
or hsMcl-1):
Seq ID No. 3
5′-GIGIAuucaugggcTIAIC-3′

(I=2′-deoxyinosine; Lowercase letters indicate 2′-O-Me/RNA; Italic: DNA)

(Mcl-1 hsRNA (double-strand hybrid siRNA),
or hsMcl-1):
Seq ID No. 4
GTGAATTCATGGGCTCATC
(hsa-miR-34a mimic):
Seq ID No. 5
uggcagugucuuagcugguugu
(precursor miR-34a stem-loop in plasmid
or cassettes):
Seq ID No. 6
ggccagcugugaguguuucuuuggcagugucuuagcugguug
uugugagcaauaguaaggaagcaaucagcaaguauacugccc
uagaagugcugcacguuguggggccc
(hsa-miR-100 mimic):
Seq ID No. 7
AACCCGUAGAUCCGAACUUGUG

1. A therapeutic nanovector comprising:
a lipoplex particle;
a CD44 antigen targeting moiety associated with the lipoplex particle; and
a therapeutic nucleic acid associated with the lipoplex particle.

2. The therapeutic nanovector of claim 1, wherein the CD44 antigen targeting moiety is anti-CD44 antibody and/or anti-CD44 scFv.

3. The therapeutic nanovector of claim 1, wherein the lipoplex particle is a liposome according to one of the following:
DOTAP/DOPE;
DDAB/DOPE;
DOTAP/Chol;
DDAB/Chol;
DOTAP/DOPE/Chol; or
DDAB/DOPE/Chol.

4. The therapeutic nanovector of claim 1, wherein the lipoplex particle is a liposome according to one of the following:
DOTAP/DOPE at about 1:1 molar ratio;
DDAB/DOPE at about 1:1 molar ratio;
DDAB/DOPE at about 1:2 molar ratio;
DOTAP/Chol at about 1:1 molar ratio;
DDAB/Chol at about 1:1 molar ratio;
DOTAP/DOPE/Chol at about 2:1:1 molar ratio; and
DDAB/DOPE/Chol at about 2:1:1 molar ratio,
wherein the molar ratios range+/−10%.

5. The therapeutic nanovector of claim 1, wherein the therapeutic nucleic acid is selected from siRNA, siRNA mimics, miRNA, miRNA mimics, shRNA, shRNA mimics, plasmid DNA encoding therapeutic agent DNA fragment, shRNA expression cassette, miRNA expression cassette, RNA, oligonucleotides, or the like.

6. The therapeutic nanovector of claim 1, wherein the therapeutic nucleic acid is selected from Bcl-2 shRNA, pshBcl-2 plasmid, Mcl-1 shRNA, pshMcl-1 plasmid, miR-34a, miR-34a mimic, or the like.

7. A method of inhibiting cancer development in a subject, the method comprising:
providing the therapeutic nanovector of claim 1; and
administering the therapeutic nanovector to the subject in an amount to inhibit cancer development.

8. The method of claim 7, comprising administering a sufficient amount of the therapeutic nanovector so as to retard progression of cancer development with the therapeutic nucleic acid.

9. The method of claim 7, wherein the therapeutic nanovector targets precancerous cells, tumor initiating cells, progenitor cells, and/or cancer stem cells having the CD44 antigen so as to inhibit cancer development with the therapeutic nucleic acid.

10. The method of claim 7, comprising administering a sufficient amount of the therapeutic nanovector so as to inhibit formation of tumorspheres with the therapeutic nucleic acid.

11. The method of claim 7, comprising administering a sufficient amount of the therapeutic nanovector so as to reduce tumor initiation and/or tumor growth with the therapeutic nucleic acid.

12. The method of claim 7, wherein the CD44 antigen targeting moiety is anti-CD44 antibody.

13. The method of claim 7, wherein the CD44 antigen targeting moiety is anti-CD44 scFv.

14. The method of claim 7, wherein the lipoplex particle is a liposome according to one of the following:
DOTAP/DOPE 1:1 molar ratio;
DDAB/DOPE 1:1 molar ratio;
DDAB/DOPE 1:2 molar ratio;
DOTAP/Chol 1:1 molar ratio;
DDAB/Chol 1:1 molar ratio;
DOTAP/DOPE/Chol 2:1:1 molar ratio; and
DDAB/DOPE/Chol 2:1:1 molar ratio,
wherein the molar ratios range+/−10%.

15. The method of claim 7, wherein the therapeutic nucleic acid is selected from siRNA, siRNA mimics, miRNA, miRNA mimics, shRNA, shRNA mimics, plasmid DNA encoding therapeutic agent, DNA fragment, shRNA expression cassette, miRNA expression cassette, RNA, oligonucleotides, or the like.

16. A method of preparing the therapeutic nanovector of claim 1, the method comprising:
providing the CD44 antigen targeting moiety with the lipoplex particle;
associating the lipid particle with the CD44 antigen targeting moiety; and
associating the therapeutic nucleic acid with the lipoplex particle.

17. The method of claim 16, comprising:
providing the components of the lipoplex particle; and
forming the lipoplex particle in the presence of the CD44 antigen targeting moiety.

18. The method of claim 17, comprising forming a lipoplex particle and CD44 antigen targeting moiety complex in the presence of serum-free media.

19. The method of claim 17, comprising forming the therapeutic nanovector to have the following: (therapeutic nucleic acid DNA)/Lipids/(CD44 antigen targeting moiety) ratio in the range of 1/(0.1-50)/(0.1-100) ug/nmol/ug.

20. The method of claim 17, comprising forming the therapeutic nanovector to have the following: (therapeutic nucleic acid miRNA or siRNA or the like)/Lipids/(CD44 antigen targeting moiety) ratio in the range of 1/(1-12)/(0.1-1.2) ug/nmol/ug.


SEQUENCE LISTING (7 sequences)

SEQ ID NO: 1 - DNA, 19 bp, Artificial Sequence
  Description of Artificial Sequence Synthetic oligonucleotide
tgaatatacacaatcaggg

SEQ ID NO: 2 - DNA, 19 bp, Artificial Sequence
  Description of Artificial Sequence Synthetic oligonucleotide
acttcatcactatctcccg

SEQ ID NO: 3 - DNA, 19 bp, Artificial Sequence
  Description of Artificial Sequence Synthetic oligonucleotide
gngnauucaugggctnanc

SEQ ID NO: 4 - DNA, 19 bp, Artificial Sequence
  Description of Artificial Sequence Synthetic oligonucleotide
gtgaattcatgggctcatc

SEQ ID NO: 5 - RNA, 22 nt, Artificial Sequence
  Description of Artificial Sequence Synthetic oligonucleotide
uggcagugucuuagcugguugu

SEQ ID NO: 6 - RNA, 110 nt, Artificial Sequence
  Description of Artificial Sequence Synthetic polynucleotide
ggccagcugugaguguuucuuuggcagugucuuagcugguuguugugagcaauaguaaggaagcaaucagcaaguauacugcccuagaagugcugcacguuguggggccc

SEQ ID NO: 7 - RNA, 22 nt, Artificial Sequence
  Description of Artificial Sequence Synthetic oligonucleotide
aacccguagauccgaacuugug